(19) United States
(12) Patent Application Publication
(10) Pub. No.: US 2014/0198895 A1
(43) Pub. Date: Jul. 17, 2014

(54) MEDICAL IMAGING SYSTEM

(71) Applicant: Konica Minolta, Inc., Tokyo (JP)

(72) Inventors: Yoshihide HOSHINO, Tokyo (JP); Junko KIYOHARA, Tokyo (JP)

(73) Assignee: Konica Minolta, Inc., Tokyo (JP)

(21) Appl. No.: 14/154,734

(22) Filed: Jan. 14, 2014

(30) Foreign Application Priority Data
Jan. 16, 2013 (JP) 2013-005047

(51) Int. Cl. A61B 6/00

(52) U.S. Cl. CPC A61B 6/4417
USPC 378/36

(57) ABSTRACT

A medical imaging system includes a radiographing apparatus and an image processing apparatus. The radiographing apparatus is provided with a Talbot or Talbot-Lau interferometer and includes an X-ray source, an X-ray detector, and a subject table. The image processing apparatus generates at least one of an X-ray absorption image, a differential phase image, and a small-angle scattering image of the subject using an image signal and a background signal obtained through subject radiographing and background radiographing, respectively. The background radiographing is performed with a member held instead of the subject. The member has a material and/or thickness to create change in energy spectrum of X-rays equivalent to change in energy spectrum of X-rays created by the subject.

### BACKGROUND OF THE INVENTION

1. Field of the Invention

The present invention relates to a medical imaging system including a radiographing apparatus provided with a Talbot interferometer or Talbot-Lau interferometer.

2. Description of Related Art

Widely-known radiographing apparatuses include conversion elements to generate electrical signals according to emitted X-rays and include an X-ray detector or flat panel detector (FPD) to read the electrical signals as image signals. Such radiographing apparatuses use, for example, a Talbot interferometer or Talbot-Lau interferometer including an X-ray source to emit X-rays to the X-ray detector and including multiple diffraction gratings etc. (see Japanese Unexamined Patent Application Publication No. 2008-200359 and WO 2011/033798, for example).

The Talbot interferometer and Talbot-Lau interferometer use Talbot effect, in which the images of a first grating having slits at regular intervals are formed at regular distances along the light travelling direction when coherent light passes through the first grating. A second grating is disposed at the position of an image of the first grating such that the second grating is slightly inclined with respect to the first grating to form moire fringes.

It is known that at least three types of reconstructed images, an X-ray absorption image, differential phase image, and small-angle scattering image, can be formed by producing images where the moire fringes appear (hereinafter referred to as moire images) through a method based on the principle of fringe scanning (see, for example, K. Hibino et al, J. Opt. Soc. Am. A, Vol. 12, (1995) p. 761-768; and A. Momose et al, J. Appl. Phys., Vol. 45, (2006) p. 5254-5262), and by analyzing the moire image using the Fourier transform (see, for example, M. Takeda et al, J. Opt. Soc. Am, Vol. 72, No. 1, (1982) p. 156).

When a moire image is produced by a radiographing apparatus provided with a Talbot interferometer or Talbot-Lau interferometer and the moire image is simply reconstructed into the three types of X-ray images, an artifact appears due to unevenness of periods and thicknesses of the gratings.

In view of this, when a subject is radiographed under a certain radiographing condition, a moire image without a subject is also produced under the same radiographing condition as that for the subject radiographing. In the image processing for reconstructing an absorption image and small-angle scattering image of the subject from the moire image, background correction is performed using the signal obtained from the moire image produced without a subject (hereinafter referred to as a background signal, which is abbreviated as a BG signal). An artifact caused by the gratings is then removed from the image signal obtained from the moire image produced with a subject.

Through such processing, an artifact caused by, for example, unevenness of periods and thicknesses of the gratings (hereinafter simply referred to as image disturbance) has been prevented from appearing in the reconstructed three types of images.

Unfortunately, the studies conducted by the inventors of the present invention have found that, when an absorption image and small-angle scattering image are generated using the BG signal obtained from the moire image without a subject and the image signal obtained from the moire image with a subject, image disturbance cannot be fully removed and sometimes remains in the absorption image and small-angle scattering image.

Such remaining image disturbance makes the absorption image and small-angle scattering image fuzzy and causes inconvenience such as oversight of a lesion part of a patient which faintly appears in an image but mixed among the image disturbance.

### SUMMARY OF THE INVENTION

The present invention has been made in view of the problems and aims to provide a medical imaging system which can surely prevent image disturbance, such as grating fringes and an artifact, from appearing in an absorption image and small-angle scattering reconstructed from a moire image(s) produced by a radiographing apparatus provided with a Talbot interferometer or Talbot-Lau interferometer.

In order to solve the problems set forth above, according to an aspect of a preferred embodiment of the present invention, there is provided a medical imaging system including: a radiographing apparatus provided with a Talbot interferometer or a Talbot-Lau interferometer, the radiographing apparatus including: an X-ray source which emits X-rays, an X-ray detector including a conversion element to generate an electrical signal according to the emitted X-rays, and reading the electrical signal generated by the conversion element, as an image signal, and a subject table to hold a subject; and an image processing apparatus which generates at least one of an X-ray absorption image, a differential phase image, and a small-angle scattering image of the subject on the basis of the image signal obtained through subject radiographing in which the subject is radiographed by the radiographing apparatus, wherein the image processing apparatus generates at least one of the X-ray absorption image, the differential phase image, and the small-angle scattering image of the subject using the image signal and a background signal obtained through the subject radiographing and background radiographing, respectively, the background radiographing being performed with a member held instead of the subject, the member having a material and/or thickness to create change in energy spectrum of X-rays equivalent to change in energy spectrum of X-rays created by the subject.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a schematic view of a medical imaging system according to an embodiment of the present invention;

FIG. 2 is a schematic plan view of a multi-slit, first grating, and second grating;

FIG. 3 illustrates the principle of a Talbot interferometer;

FIG. 4A is an example absorption image (photograph) obtained by performing background correction on an image signal using a BG signal obtained through a conventional background radiographing;

FIG. 4B is an example small-angle scattering image (photograph) obtained by performing background correction on an image signal using a BG signal obtained through a conventional background radiographing;

FIG. 5 is a graph showing that, when a subject is present, the energy spectrum of X-rays shifts to the high energy side compared to when a subject is not present;

FIG. 6 is a graph showing that performing background radiographing with a member changes the energy spectrum of X-rays into a spectrum equivalent to the energy spectrum of X-rays obtained when a subject is present;

FIG. 7A is an example absorption image (photograph) obtained by performing background correction on an image signal using a BG signal obtained through background radiographing with a member;

FIG. 7B is an example small-angle scattering image (photograph) obtained by performing background correction on an image signal using a BG signal obtained through background radiographing with a member;

FIG. 8A is a photograph showing that a relatively sharp absorption image is obtained when a body movement of a subject is small;

FIG. 8B is a photograph showing that a relatively sharp differential phase image is obtained when a body movement of a subject is small;

FIG. 9A is a photograph showing that a blurred absorption image is obtained when a body movement of a subject is large;

FIG. 9B is a photograph showing that a blurred differential phase image is obtained when a body movement of a subject is large;

FIG. 10 illustrates pixels corresponding to the location of a bone edge found in an absorption image etc.;

FIG. 11 is an example differential phase image (photograph) of a joint showing an edge of a joint cartilage;

FIG. 12 illustrates pixels corresponding to the location of a bone edge and pixels corresponding to a cartilage edge in a differential phase image;

FIG. 13A illustrates that the distribution of frequency F of a histogram is wide when a body movement of a subject is small;

FIG. 13B illustrates that the distribution of frequency F of a histogram is narrow when a body movement of a subject is large;

FIG. 14 illustrates the case in which a body movement of a subject occurs between the m^{th} subject radiographing and the (m+1)^{th} subject radiographing; and

FIG. 15 illustrates division of M image signals into two groups G1 and G2, and translation of the image signals belonging to the group G2 relative to the image signals belonging to the group G1.

### DESCRIPTION OF THE PREFERRED EMBODIMENTS

Embodiments of a medical imaging system according to the present invention will now be described with reference to the attached drawings.

### [Configuration of Medical Imaging System]

As described above, a medical imaging system according to the invention includes a radiographing apparatus provided with a Talbot interferometer or Talbot-Lau interferometer.

The Talbot effect, which is the principle of a Talbot interferometer etc., refers to a phenomenon in which when coherent light passes through a first grating (G1 grating) with slits at regular distances, the image of the grating is formed at regular distances along the direction of the propagating light. The formed images are called self-images. The Talbot interferometer has a second grating (G2 grating) at the location of a self-image, and forms moire fringes by slightly inclining the second grating with respect to the first grating.

Positioning an object in front of the first grating disrupts the moire fringes. A medical imaging system including a radiographing apparatus provided with a Talbot interferometer produces images including moire fringes (hereinafter referred to as moire images) obtained through irradiations with coherent X-rays with and without a subject in front of the first grating. The system then analyzes these images to produce a reconstructed image of the subject. The configuration of the present invention concerning these processes is described later in detail.

Talbot-Lau interferometers are also known which have a multi-slit grating (G0 grating) between the X-ray source and the first grating. A medical imaging system including a radiographing apparatus provided with a Talbot-Lau interferometer basically has a similar structure to a system provided with a Talbot interferometer except that it contains a multi-slit grating to use a high-output incoherent X-ray source which can increase radiation dose per unit time, for example.

As described above, a radiographing apparatus provided with a Talbot interferometer or Talbot-Lau interferometer, which produces moire images, can produce at least three types of reconstructed images: an X-ray absorption image, differential phase image, and small-angle scattering image, by producing moire images with a scheme based on the principle of fringe scanning or by analyzing the moire image(s) with Fourier transform.

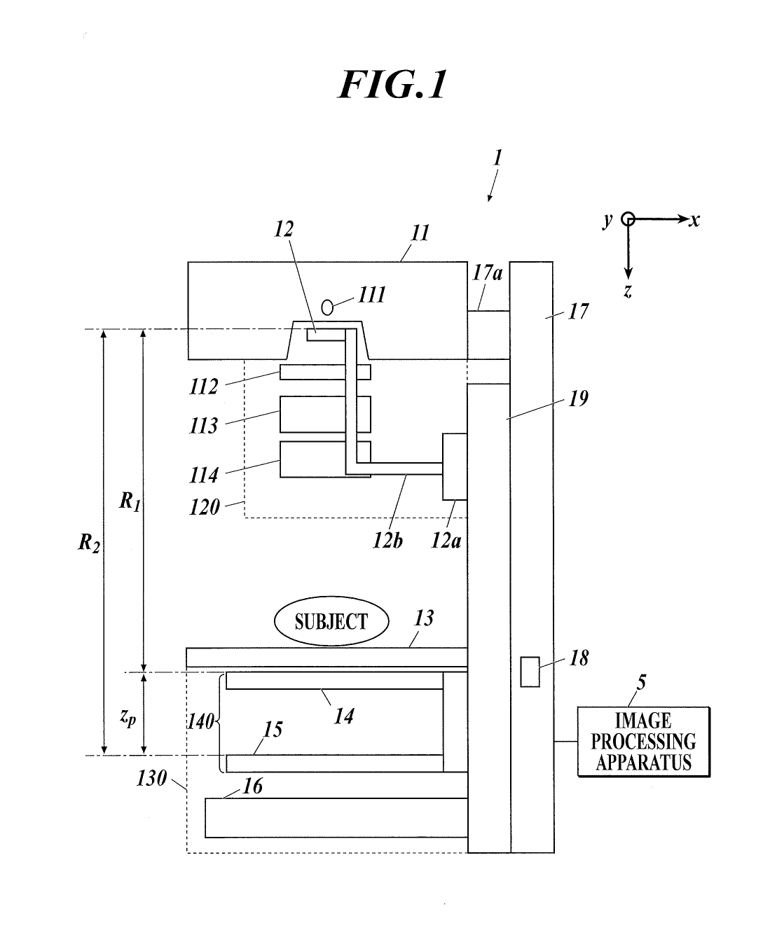

The configuration of the medical imaging system according to this embodiment will now be briefly described. FIG. 1 schematically illustrates the medical imaging system of this embodiment.

As shown in FIG. 1, the medical imaging system includes a radiographing apparatus 1 and an image processing apparatus 5. In FIG. 1, the radiographing apparatus 1 is provided with a Talbot-Lau interferometer. In the following description, the radiographing apparatus 1 is provided with the Talbot-Lau interferometer. The invention is also applicable to a radiographing apparatus provided with a Talbot interferometer. The following description is also applicable to a radiographing apparatus provided with a Talbot interferometer.

The image processing apparatus 5 generates reconstructed images, i.e., an X-ray absorption image, differential phase image, and small-angle scattering image of the subject from moire images produced by the radiographing apparatus 1. As described later, the image processing apparatus 5 does not necessarily have to generate all of the absorption image, differential phase image, and small-angle scattering image. The image processing apparatus 5 generates at least one of the three types of images. The process in the image processing apparatus 5 will be described later in detail.

### [Configuration of Radiographing Apparatus]

As shown in FIG. 1, the radiographing apparatus 1 of the medical imaging system includes an X-ray source 11; a first covering unit 120 containing a multi-slit 12; a second covering unit 130 containing a subject table 13, a first grating 14, a second grating 15, and an X-ray detector 16; a support 17; a main body 18; and a base 19.

The radiographing apparatus 1 in FIG. 1 is upright. The X-ray source 11 (having a focal point 111), the multi-slit 12, the subject table 13, the first grating 14, the second grating 15, and the X-ray detector 16 are disposed in sequence in the z direction, i.e., the direction of the gravity. The z-direction is the direction of illumination axis of X-rays emitted from the X-ray source 11.

In FIG. 1, the first covering unit 120 contains an adjuster 12a, a mounting arm 12b, an additional filter 112, an irradiation field diaphragm 113, and an irradiation field lamp 114. The second covering unit 130 contains a grating assembly 140 including the first grating 14 and the second grating 15.

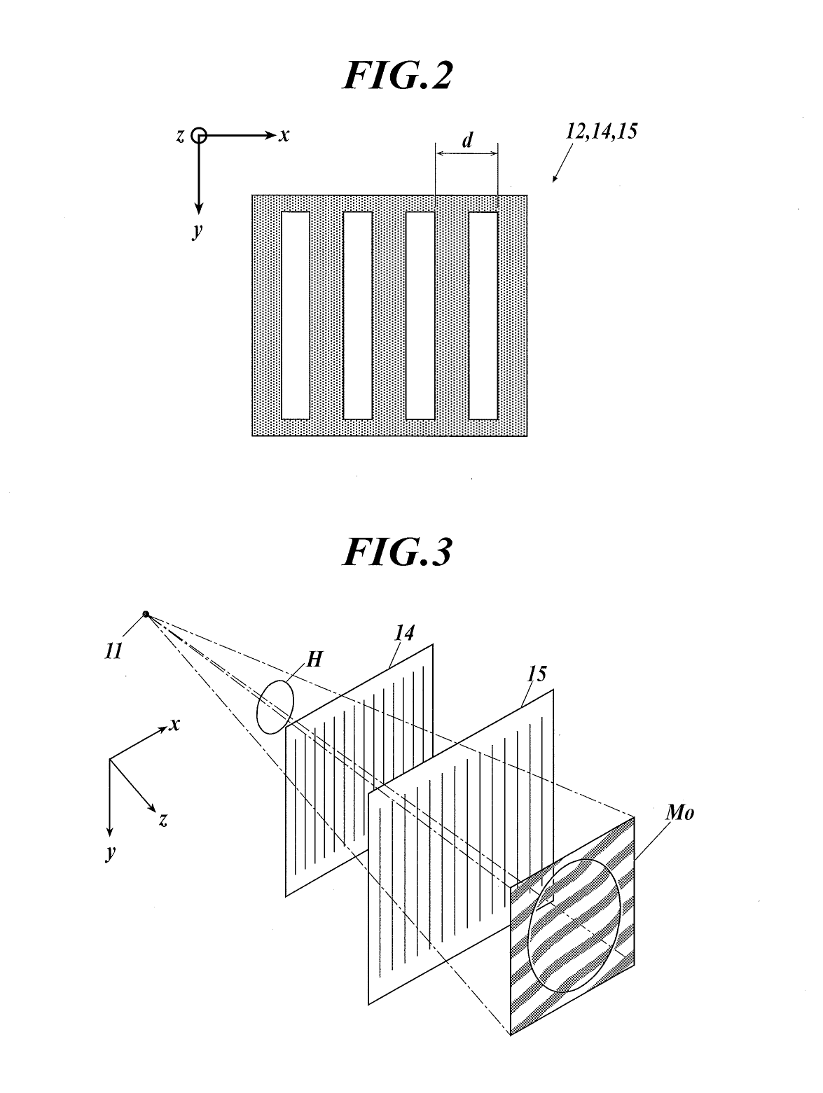

In this embodiment, the components in the first and second covering units 120 and 130 are each protected with a covering material (not shown). In the radiographing apparatus 1 producing moire images by fringe scanning, the second covering unit 130 is provided with a mechanism (not shown) for moving the second grating 15 in a given direction (the x direction in FIGS. 1 and 2), for example.

The adjuster 12a is used for fine adjustment of the location of the multi-slit 12 along the x, y, and z directions and the rotational angle of the multi-slit 12 around the x, y, and z axes. The adjuster 12a is not essential if the multi-slit 12 can be accurately fixed to the support 19. In FIG. 1, the reference numeral 17a is a cushion connecting the X-ray source 11 and the support 17.

As illustrated in FIG. 2, the multi-slit 12 (G0 grating), the first grating 14 (G1 grating), and the second grating 15 (G2 grating) are diffraction gratings provided with plural slits arranged in the x direction orthogonal to the z direction, i.e., the direction of the illumination axis of X-rays. Refer to, for example, WO 2011/033798 for the material or process for forming these gratings.

As shown in FIG. 2, the multi-slit 12, the first grating 14, and the second grating 15 have inter-slit distances d (d₀, d₁, and d₂, respectively). As shown in FIG. 1, R₁ is the distance between the multi-slit 12 and the first grating 14, R₂ is the distance between the multi-slit 12 and the second grating 15, and zₚ is the distance between the first grating 14 and the second grating 15. Expressions (1) to (4) or similar conditions hold (see W. Yashiro et al., Efficiency of capturing a phase image using cone-beam X-ray Talbot interferometry. Opt. Soc. Am., 25, 2025, 2008.).

zₚ=pd₁·αd₂/λ (1)

d₂=R₂d₁/(R₁α) (2)

R₁/d₀=zₚ/d₂ (3)

1/d₀=α/d₁−1/d₂ (4)

Here, p and α are Talbot order and Talbot constant, respectively, which vary depending on the type of the first grating 14. Typical examples are listed below. In this table, n is a positive integer.

TABLE 1

| | Π/2 SHIFT | | ABSORPTION |
|---|---|---|---|
| | DIFFRACTION | ΠSHIFT DIFFRACTION | DIFFRACTION |
| | GRATING | GRATING | GRATING |
| p | (2n − 1)/2 | (2n − 1)/8 | n |
| α | 1 | 2 | 1 |

Under the above conditions, self-images formed by X-rays passing through the slits of the multi-slit 12 and the first grating 14 can be superimposed on each other on the second grating 15.

### [Principles of Talbot Interferometer and Talbot-Lau Interferometer]

The Principle common to Talbot interferometer and Talbot-Lau interferometer will now be described. As shown in FIG. 3, when X-rays from the X-ray source 11 pass through the first grating 14, the X-rays produce images formed at regular distances along the z direction. These images are called self-images. Such a phenomenon in which self-images are formed at regular distances along the z direction is called Talbot effect.

The second grating 15 is located at the position where a self-image of the first grating 14 appears. In addition, a direction in which the slits of the second grating 15 extend (i.e., the y direction in FIG. 2) is slightly inclined with respect to the direction in which the slits of the first grating 14 extend. Thus, a moire image (shown as Mo in FIG. 3) appears on the second grating 15.

FIG. 3 depicts a moire image No as being away from the second grating 15 to avoid any confusion which may be caused by depicting a moire image Mo on the second grating 15. In practice, a moire image Mo is formed on and downstream of the second grating 15. In FIG. 3, the subject H present between the X-ray source 11 and the first grating 14 is reflected in the moire image Mo. If the subject H is not present, only moire fringes appear.

The subject H present between the X-ray source 11 and the first grating 14 may shift the phase of X-rays, depending on the type of the subject. Thus, as shown in FIG. 3, the fringes in the moire image No are disturbed around the frame of the subject. The disturbed moire fringes are detected through processing of the moire image Mo. The image of the subject is then reconstructed. This is the principle of the Talbot interferometer.

### [Other Configurations in Radiographing Apparatus]

Other configurations in the radiographing apparatus 1 shown in FIG. 1 will now be described. The subject table 13 holds a subject. The X-ray detector 16 includes a two-dimensional array of conversion elements (not shown) to generate electrical signals according to emitted X-rays and reads the electrical signals generated by the conversion elements, as image signals.

As the distance between the X-ray detector 16 and the second grating 15 increases, blurring of a moire image Mo produced by the X-ray detector 16 increases. To avoid such a phenomenon, the X-ray detector 16 is preferably fixed to the support 19 so as to be in contact with the second grating 15.

The X-ray detector 16 is a flat panel detector (FPD), for example. The FPD may be of an indirect type that converts X-rays into electrical signals through scintillator with photoelectric elements or of a direct type that directly converts X-rays into electrical signals. The X-ray detector 16 may be any FPD or any other image capturing unit such as a charge coupled device (CCD) or an X-ray camera.

The main body 18 is connected to the X-ray source 11, the X-ray detector 16, and other components and controls irradiation with X-rays from the X-ray source 11. The main body 18 transmits a moire image Mo generated by the X-ray detector 16 to the image processing apparatus 5. Alternatively, the main body 18 generates a moire image Mo from electrical signals read by the X-ray detector 16 and transmits the moire image Mo to the image processing apparatus 5.

In addition, the main body 18 comprehensively controls the radiographing apparatus 1. Not surprisingly, the main body 18 may contain any appropriate unit or device, such as an input unit, a display unit, or a storage unit.

[Configuration Etc. Of Image Processing Apparatus]

The configuration etc. of the image processing apparatus 5 in the medical imaging system according to this embodiment will now be described. In this embodiment, as described above, the image processing apparatus 5 is configured to generate the reconstructed images, i.e., an X-ray absorption image, differential phase image, and small-angle scattering image of a subject from a moire image Mo produced by the radiographing apparatus 1. The image processing apparatus 5 does not necessarily have to generate all these three reconstructed images.

In this embodiment, the image processing apparatus 5 is a computer with a bus connected to a central processing unit (CPU), a read only memory (ROM), a random access memory (RAM), an input/output interface, and other components, which are not shown in the drawing. The radiographing apparatus 1 and the image processing apparatus 5 are connected via a network.

In response to reception of multiple moire images Mo produced by fringe scanning in the radiographing apparatus 1 provided with a Talbot interferometer or Talbot-Lau interferometer, the image processing apparatus 5 reconstructs an X-ray absorption image, differential phase image, and small-angle scattering image using the image signals of the moire images.

An approach for imaging in the radiographing apparatus 1 without fringe scanning include increasing the angle between the directions of the first and second gratings 14 and 15, transmitting the image signal of a produced moire image Mo with finer moire fringes from the radiographing apparatus 1 to the image processing apparatus 5, and analyzing the transmitted image signal in the image processing apparatus 5 by Fourier transform. The approach allows an X-ray absorption image, differential phase image, and small-angle scattering image to be generated in a similar manner to the above-stated case.

[Basic Procedure Up to Generation of Absorption Image Etc. In Medical Imaging System]

The following is a conventional procedure from radiographing to generation of an absorption image etc. based on a moire image Mo by the image processing apparatus in the medical imaging system. The following procedure is basically followed in the medical imaging system according to this embodiment.

Specifically, a subject held on the subject table 13 is irradiated with X-rays using the above-described radiographing apparatus 1, and a moire image Mo is produced by the X-ray detector 16 (hereinafter referred to as subject radiographing).

When using the fringe scanning for radiographing, a plurality of moire images Mo are produced while the second grating 15, for example, (see FIGS. 1 and 2) is shifted in a given direction (i.e., x direction) as described above. When the Fourier transform is used for the analysis of a moire image(s) Mo by the image processing apparatus 5, one or a given number of moire images Mo are produced.

Before or after the subject radiographing, background radiographing is performed under the same radiographing condition as that for the subject radiographing. Specifically, irradiation is made with no subject held on the subject table 13 and a moire image Mo is produced with the X-ray detector 16.

Such a moire image Mo obtained through the background radiographing with no subject is hereinafter referred to as a BG moire image Mb to be distinguished from the moire image Mo with a subject. The signal obtained from the BG moire image Mb is hereinafter referred to as a background signal, which is abbreviated to a BG signal.

When the fringe scanning is used for the background radiographing, a plurality of BG moire images Mb are produced while the second grating 15, for example, is shifted in a given direction; and when the Fourier transform is used for the analysis of a BG moire image(s) Mb by the image processing apparatus 5, one or a given number of BG moire images Mb are produced, as in the case of the subject radiographing.

After the completion of the subject radiographing and background radiographing, all the image signal (s) of the moire image(s) Mo obtained through the subject radiographing and all the BG signal (s) of the BG moire image(s) Mb obtained through the background radiographing are transmitted to the image processing apparatus 5.

The image processing apparatus 5 calculates the pixel values for an absorption image, differential phase image, and small-angle scattering image on the basis of the image signal and the BG signal to reconstruct the absorption image etc. The image signal for each pixel (i.e., each conversion element; the same will apply to the following descriptions) of a moire image Mo obtained through subject radiographing is indicated as I_{S} (x,y); while the BG signal of each pixel of a BG moire image Mb obtained through background radiographing is indicated as I_{BG} (x,y).

The image processing apparatus 5 analyzes a plurality of moire images Mo and BG moire images Mb when the fringe scanning is used. The following descriptions are for the case of the fringe scanning. The descriptions, however, also apply to the case in which one or a given number of moire images Mo and BG moire images Mb are processed through the Fourier transform.

The image processing apparatus 5 approximates each of an image signal I_{S} (x,y) and BG signal I_{BG} (x,y) by the sum of at least the direct-current (DC) component I₀ and the first-order amplitude component I₁ of moire fringes. In the following expressions, x and y represent a pixel position, and M represents the number of times of fringe scanning. Further, the grating moves by 1/M of the gross movement at one time. Each of the results represents the signal at the k^{th} grating position.

I_{S}(x,y,k)=I₀(E_{S0},x,y)+I₁(E_{S1},x,y)×cos 2π(yθ/d₂+k/M) (5)

I_{BG}(x,y,k)=I₀(E_{BG0},x,y)+I₁(E_{BG1},x,y)×cos 2π(yθ/d₂+k/M) (6)

E_{S0} is the value representing the energy spectrum of X-rays which have passed through the gratings and subject, and E_{BG0} is the value representing the energy spectrum of X-rays which have passed through the gratings. E_{S0} and E_{BG0} are, for example, the average values or peak values of the X-rays. Further, E_{S1} and E_{BG1} are each an energy value representing the amplitude of moire fringes determined on the basis of the energy spectrum of X-rays and the energy set at the time of designing of the thicknesses and positions of the gratings. More specifically, the energy spectrum for E_{S1} is the spectrum of X-rays which have passed through the gratings and subject, while the energy spectrum for E_{BG1} is the spectrum of X-rays which have passed through the gratings.

Further, θ represents a relative angle formed by the first grating 14 and the second grating 15; d₂ represents a pitch d of the second grating 15 as described above (see FIG. 2); ζ represents a coefficient determined depending on a grating and its position; and φ_{X} represents a refraction angle of X-rays created by a subject.

When an image signal I_{S} (x,y) and BG signal I_{BG} (x,y) are expressed as described above, the pixel values I_{AB} (x,y), I_{DP} (x,y), and I_{V} (x,y) of an absorption image, differential phase image, and small-angle scattering image, respectively, are obtained through the following calculations.

I_{AB}(x,y)=I₀(E_{S0},x,y)/I₀(E_{BG0},x,y) (7)

I_{DP}(x,y)=(yθ/d₂+ζφ_{X}(E_{S1},x,y)−yθ/d₂))/ζ (8)

∴I_{DP}(x,y)=φ_{X}(E_{S1},x,y) (9)

I_{V}(x,y)=(I₁(E_{S1},x,y)/I₀(E_{S0},x,y))/(I₁(E_{BG1},x,y)/I₀(E_{BG0},x,y)) (10)

A conventional procedure for generating an absorption image etc. from a moire image Mo etc. by the image processing apparatus 5 is basically as described above. Specifically, for calculating the pixel value I_{AB} (x,y) of an absorption image, the DC component I₀ of moire fringes of the image signal I_{S} (x,y) expressed by the expression (5) is divided by the I₀ of the BG signal I_{BG} (x,y) expressed by the expression (6).

The pixel value I_{DP} (x,y) of a differential phase image is obtained as a refraction angle φ_{X} of X-rays created by a subject. Furthermore, for calculating the pixel value I_{V} (x,y) of a small-angle scattering image, the ratio between the first-order amplitude component (I₁) and the DC component (I₀) of moire fringes of the image signal I_{S} (x,y) is divided by that of the BG signal I_{BG} (x,y).

Specifically, when generating at least an absorption image and small-angle scattering image, I₀ and I₁ of the image signal I_{S} (x,y) is divided by I₀ and I₁ of the BG signal I_{BG} (x,y), as shown in expressions (7) and (10).

Thus, the conventional method of generating an absorption image etc. makes the components of artifact or image disturbance offset with each other, which artifact appears in each of the image signal I_{S} (x,y) and BG signal I_{BG} (x,y) due to unevenness of periods and thicknesses of the gratings. In this way, the conventional method prevents image disturbance from appearing in generated absorption images and small-angle scattering images etc.

As shown in the expression (8), for a differential phase image, image disturbances are offset with each other by subtracting the variable of the cosine function of the BG signal I_{BG} (x,y) from that of the image signal I_{S} (x,y). As shown in the expression (9), since the image signal is obtained as the refraction angle φ_{X} of X-rays created by a subject, it is thought that a differential phase image is almost free from the influence of image disturbance. The differential phase image, therefore, may basically by reconstructed on the basis of the expression (8) using the same BG image as that for an absorption image and small-angle scattering image.

[Phenomenon of Image Disturbance Remaining in Absorption Image Etc. Generated with Conventional Method]

The studies conducted by the inventors of the present invention, however, show that image disturbance cannot be fully removed from at least an absorption image and small-angle scattering image when performing the division and subtraction on the components of the image signal I_{S} using the corresponding components of the BG signal I_{BG} obtained from a BG moire image Mb produced through the background radiographing. In other words, image disturbance remains in an absorption image and small-angle scattering image in some cases.

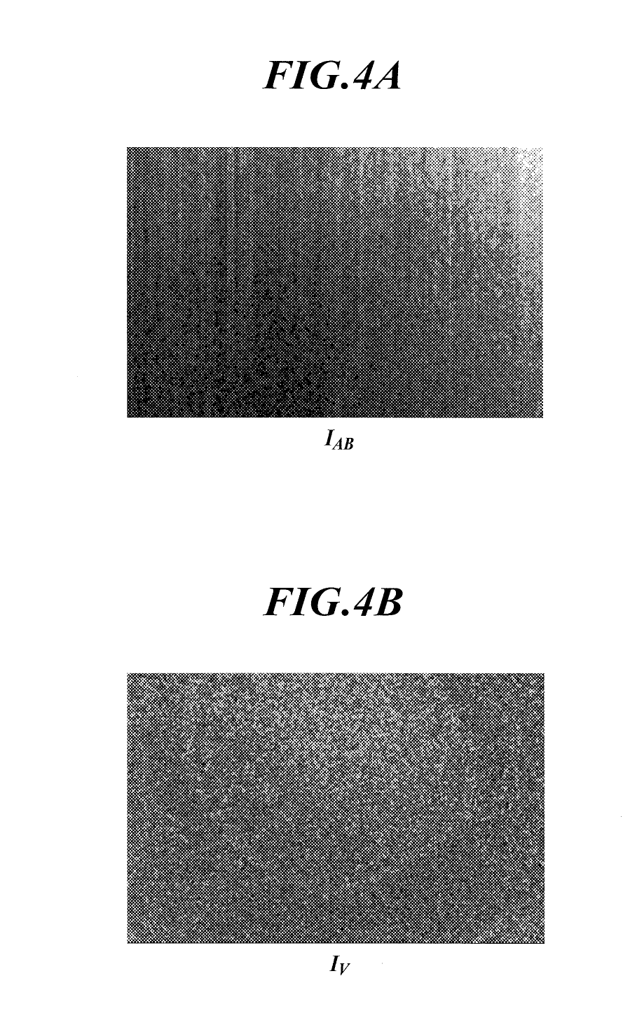

FIGS. 4A and 4B show an example absorption image I_{AB} (see FIG. 4A) and an example small-angle scattering image I_{V} (see FIG. 4B) obtained as described above (see the expressions (7) and (10)) by performing background correction on the image signal I_{S} using the BG signal I_{BG}. The image signal I_{S} is obtained through radiographing with an aluminum plate (as a subject) having a thickness of 1.3 mm. The BG signal I_{BG} is obtained through background radiographing without the 1.3-mm-thickness aluminum plate. Both of the radiographing is performed under the condition of the tube voltage of 40 kV (with 1.0-mm AL added).

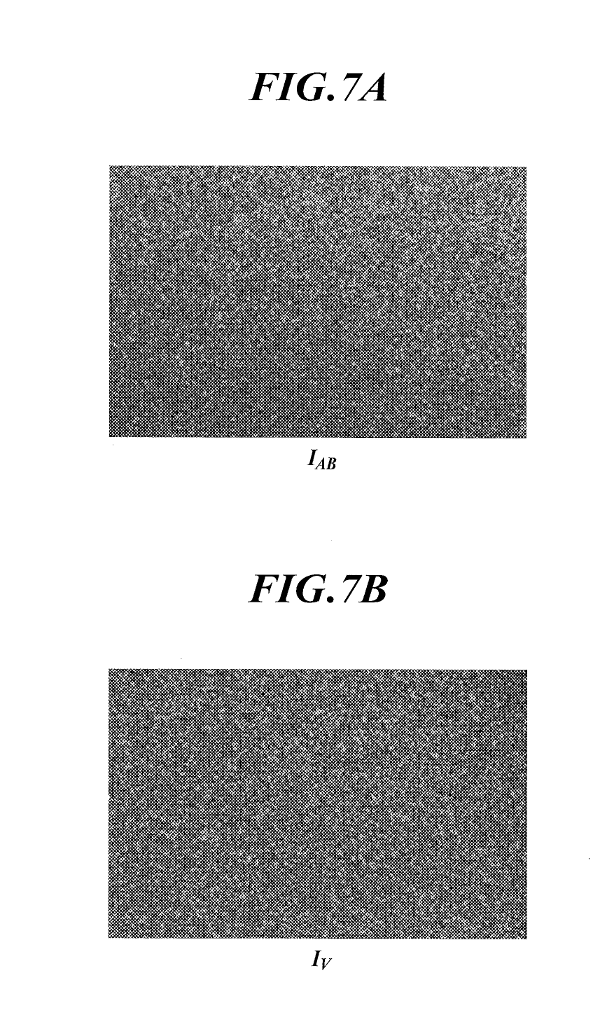

In the examples shown in FIGS. 4A and 4B and FIGS. 7A and 7B (described later), the subject or 1.3-mm-thickness aluminum plate covers the entire area of a moire image Mo (not shown). The edge of the aluminum plate (i.e., the edge part) does not appear in the moire image Mo, absorption image I_{AB}, and small-angle scattering image I_{V}.

In the absorption image I_{AB} shown in FIG. 4A, for example, image disturbance remains to be removed, which is thought to be due to unevenness of thicknesses of the first grating 14 and the second grating 15. In the small-angle scattering image I_{V} shown in FIG. 4B, for example, a circular pattern, i.e., image disturbance, remains to be removed, which is thought to be due to unevenness of thickness of the first grating 14.

In other words, in the above-described examples, the processing (see the expressions (7) and (10)) fails to offset the components of image disturbances appearing in the image signal I_{S} (x,y) and the BG signal I_{BG} (x,y) with each other. As seen above, it has been found that the conventional method may not fully remove image disturbance from at least an absorption image I_{AB} and small-angle scattering image I_{V} reconstructed on the basis of a moire image Mo and BG moire image Mb.

[Causes Etc. Of Phenomenon of Remaining Image Disturbance]

According to the studies conducted by the inventors of the present invention, such a phenomenon is thought to be due to the following causes.

In the background correction for an absorption image I_{AB}, the DC components I₀ of moire fringes of the image signal I_{S} (x,y) and BG signal I_{BG} (x,y) include E_{S0} and E_{BG0}, respectively, as shown in the expression (7). In the background correction for a small-angle scattering image I_{V}, the first-order amplitude components I₁ of moire fringes of the image signal I_{S} (x,y) and BG signal I_{BG} (x,y) include E_{S1} and E_{BG1} in addition to E_{S0} and E_{BG0}, respectively, as shown in the expression (10).

While E_{BG0} and E_{BG1} are values dependent on the energy spectrum of X-rays which have passed through only the gratings, E_{S01} and E_{S1} are values dependent on the energy spectrum of X-rays which have passed through both the gratings and a subject, as described above. When X-rays pass through a subject, the subject scatters the components mainly with a long wavelength (i.e., low-energy components).

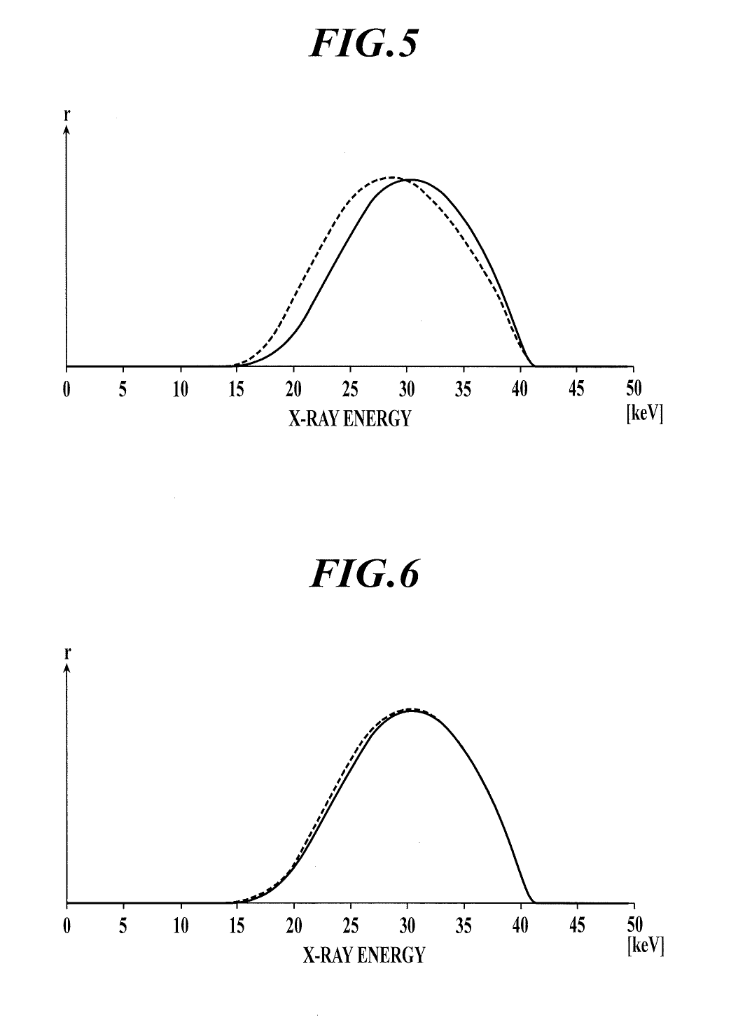

The energy of X-rays reaching the first grating 14 (see FIG. 3) thus varies in spectrum between the case with a subject (i.e., the subject radiographing) and the case without a subject (i.e., the background radiographing) as shown in, for example, FIG. 5. Specifically, the average or peak value of the energy spectrum of X-rays shifts to the high energy side in the case with a subject compared to the case without a subject.

FIG. 5 shows the results of calculations of the energy spectrum of X-rays on the side of the X-ray incidence plane of the first grating 14 based on literature data with and without a subject. The calculations are performed using a tungsten tube with the tube voltage of 40 kV (with 2.5-mm AL added). The subject contains 50% of mammary gland and 50% of fat with a uniform thickness of 45 mm. The solid and broken lines represent the cases with and without a subject, respectively. FIGS. 5 and 6 do not show the absolute values of the amount of transmitted X-rays but show the X-rays spectrum distribution.

The difference in X-ray energy spectrum between the cases with and without a subject (i.e., the cases of subject radiographing and background radiographing, respectively) leads to the difference in proportion of the energy of X-rays having a wavelength aimed by the first grating 14 in the X-ray energy spectrum and in the transmittance of X-rays through the second grating 15. It is thought, therefore, that there is a difference in the intensity distribution of self-images formed by the X-rays passing through the first grating 14 and in the distribution of the transmittance of X-rays through the second grating 15 between the cases with and without a subject.

This makes the degrees of image disturbances appearing in the image signal I_{S} (x,y) and BG signal I_{BG} (x,y) different from each other. As a result, the divisions as shown in the expressions (7) and (10) fail to offset the components of image disturbances with each other. This is thought to be one of the causes of image disturbance remaining in an absorption image I_{AB} and small-angle scattering image I_{V} as shown in FIGS. 4A and 4B.

Conversely, making the energy spectrums of X-rays reaching the first grating 14 the same between the subject radiographing (with a subject) and the background radiographing (without a subject) results in a uniform ratio, between the cases with and without a subject, in the intensity distribution of the self-images formed by the X-rays passing through the first grating 14 and in the distribution of the transmittance of X-rays through the second grating 15.

This makes the degree of image disturbance included in the image signal I_{S} (x,y) the same as that in the BG signal I_{BG} (x,y). Thus, the calculations in accordance with the expressions of (7) and (10) allow offset of the image disturbances with each other, removing the image disturbance from an absorption image I_{AB} and small-angle scattering image I_{V}.

A part of X-rays (mainly long-wavelength components) passing through a subject is absorbed by the subject, causing change in X-ray energy spectrum. In view of this, making an irradiation with a member held which absorbs as much X-rays as the subject, such as an acrylic, for the background radiographing allows the energy spectrum of X-rays reaching the first grating 14 in the background radiographing to be equivalent to that in the subject radiographing.

FIG. 6 shows the results of calculations of the energy spectrum of X-rays reaching the first grating 14 based on literature data. The results are obtained with an acrylic plate having a uniform thickness of 40 mm held in the background radiographing under the same condition as for FIG. 5, i.e., using a tungsten tube with the tube voltage of 40 kV (with 2.5-mm AL added). Similarly to the above, the solid line represents the case of subject radiographing with a subject, and the broken line represents the case of background radiographing with the acrylic plate.

According to the results, it has been found that, in the background radiographing, placing a member such as the acrylic plate changes the energy spectrum of X-rays reaching the first grating 14. It has also been found that changing the material (e.g., changing the acrylic plate to an aluminum plate) and/or changing the thickness of the member changes the degree of change in energy spectrum of X-rays, although not shown in FIG. 6.

The energy spectrum of X-rays reaching the first grating 14 through the member in the background radiographing is made equivalent to that in the subject radiographing. It has been found that, when an absorption image I_{AB} and small-angle scattering image I_{V} are generated on the basis of the image signal I_{S} (x,y) and BG signal I_{BG} (x,y) calculated from a moire image Mo and BG moire image Mb, respectively, produced in such a state, image disturbance does not remain in the absorption image I_{AB} and small-angle scattering image I_{V} as shown in FIGS. 7A and 7B.

FIGS. 7A and 7B show an example absorption image I_{AB} (see FIG. 7A) and an example small-angle scattering image I_{V} (see FIG. 7B) based on the image signal I_{S} and BG signal I_{BG} obtained through the subject radiographing and background radiographing, respectively. The subject radiographing is performed using an aluminum plate (as a subject) having a thickness of 1.3 mm, while the background radiographing is performed using an aluminum plate (as a member) having a thickness of 1.3 mm. Both of the radiographing is performed under the condition of the tube voltage of 40 kV (with 1.0-mm AL added) similarly to the case shown in FIGS. 4A and 4B.

The studies conducted by the inventors of the present invention have found that one of the causes of image disturbance remaining in an absorption image I_{AB} and small-angle scattering image I_{V} generated through the conventional method is the difference in energy spectrum of X-rays reaching the first grating 14 between when a subject is present (i.e., the case of subject radiographing) and when a subject is not present (i.e., the case of background radiographing) (see, for example, FIG. 5).

In the background radiographing, a BG moire image Mb is produced with a member held instead of a subject, the material and/or thickness of the member being designed to make the change in energy spectrum of X-rays equivalent to the change in energy spectrum of X-rays that would be created by a subject (see FIG. 6).

Performing the conventional calculation processing on the BG signal I_{BG} (x,y) obtained as described above and on the image signal I_{S} (x,y) obtained through the subject radiographing, and further performing the background correction according to the expressions of (7) and (10) can surely remove image disturbance from an obtained absorption image I_{AB} (see FIG. 7A) and small-angle scattering image I_{V} (see FIG. 7B), surely preventing image disturbance from appearing in these images.

The term “equivalent” includes the case in which the energy spectrum of X-rays in the subject radiographing is almost identical to that in the background radiographing, as well as the case in which they are completely identical to each other. Further, the state in which “the energy spectrums of X-rays are almost identical to each other” means the state in which image disturbance cannot visually recognized in an absorption image I_{AB} and small-angle scattering image I_{V} obtained through the above-described calculation processing on the image signal I_{S} (x,y) and BG signal I_{BG} (x,y) obtained through the subject radiographing and background radiographing.

[Configuration Etc. Of Medical Imaging System According to Present Invention]

In the medical imaging system according to this embodiment, the image processing apparatus 5 performs image processing, i.e., calculation processing etc. represented by the expressions (5)-(10) similarly to the conventional medical imaging system as described above. The medical imaging system according to this embodiment, however, is different from the conventional one in that the radiographing apparatus 1 performs background radiographing with the member having the material and/or thickness to create change in energy spectrum of X-rays equivalent to the change in energy spectrum of X-rays created by a subject, instead of the conventional background radiographing in which nothing is held between the X-ray source 11 and the first grating 14.

The material and/or thickness of the member to be held should be appropriately selected in order that the energy spectrum of X-rays which have passed through the member in the background radiographing is equivalent to the energy spectrum of X-rays which have passed through a subject in the subject radiographing (i.e., in order to obtain the results of FIG. 6 instead of FIG. 5).

The specific configurations etc. to achieve it are described below with some examples. The behavior of the medical imaging system according to this embodiment is also described.

For ease of explanation, the previously-given descriptions are made on the premise that a subject and member are so large that their edges (edge parts) do not appear in a moire image Mo, BG moire image Mb, absorption image I_{AB} and small-angle scattering image I_{V}. The descriptions given below are on the same premise for ease of explanation.

In an actual subject radiographing, however, there are many cases in which an edge of a subject appears in a moire image Mo, i.e., cases in which a moire image Mo includes both the area of subject and the area of background (e.g., the state shown in FIG. 3). In such cases, the energy spectrum of X-rays reaching the first grating 14 is different between a portion on the first grating 14 corresponding to the area within the subject and a portion on the first grating 14 corresponding to the background area outside the subject area in a moire image Mo.

Specifically, the energy spectrum of X-rays exhibits the spectrum represented by the solid line in FIG. 5 for the portion on the first grating 14 corresponding to the area within the subject; while the energy spectrum of X-rays exhibits the spectrum represented by the broken line in FIG. 5 for the portion on the first grating 14 corresponding to the background area outside the subject area.

It is not the background area but the subject area that should be prevented from being subject to image disturbance, such as grating fringes and an artifact, in an absorption image I_{AB} and small-angle scattering image I_{V}.

For this reason, when a moire image Mo includes both a subject area and a background area, an area of interest including the subject area is set in the moire image Mo. The material and/or thickness of a member is preferably selected so that the energy spectrum of X-rays obtained at the portion on the first grating 14 corresponding to the area of interest is equivalent to the spectrum obtained in the subject radiographing, according to the examples set forth below.

### Example 1

The following is the simplest method to make the energy spectrum of X-rays which have passed through a member in background radiographing equivalent to the energy spectrum of X-rays which have passed through a subject in subject radiographing.

Specifically, in subject radiographing, the energy spectrum of X-rays is actually measured at a position immediately under the subject table 13 (see FIG. 1) or at the subject-side face of the first grating 14 (i.e., the upper face in FIG. 1). After the subject radiographing, multiple-time background radiographing is performed with members made of different materials and/or having different thicknesses held on the subject table 13. The energy spectrum of X-rays is measured at the same position as that described above in each background radiographing.

The member having the material and/or thickness is selected so as to make the energy spectrum of X-rays to be equivalent to that in the subject radiographing. Instructions are then given to the image processing apparatus 5 to perform the calculation processing using the BG signal I_{BG} (x,y) obtained with the selected member held. The image processing apparatus 5 performs the calculation processing using the BG signal I_{BG} (x,y) obtained with the instructed member and using the image signal I_{S} (x,y) obtained through subject radiographing to generate an absorption image I_{AB} and small-angle scattering image I_{V} of the subject.

Such a configuration enables the calculation processing using the BG signal I_{BG} (x,y) (i.e., the instructed BG signal I_{BG} (x,y) in this case) obtained through the background radiographing with the member having the material and/or thickness to create change in energy spectrum of X-rays equivalent to the change in energy spectrum of X-rays created by a subject, and using the image signal I_{S} (x,y) obtained through the subject radiographing. This achieves generation of an absorption image I_{AB} and small-angle scattering image I_{V} from which image disturbance has been surely removed.

### Example 2

The above-described Example 1 advantageously enables selection of a member with high accuracy since the subject radiographing and background radiographing can be performed under the same conditions (including the temperatures of the gratings).

It is inefficient, however, to change the material and/or thickness of a member for the background radiographing each time the subject radiographing is performed. Moreover, making an irradiation for every background radiographing leads to waste of electrical power and shorter life of the X-ray source 11. The method of Example 1 might not be practical.

As a more practical method, the material and/or thickness of the member to be held in background radiographing performed for subject radiographing may be determined and notified to a radiation technologist etc.

What affects the change in energy spectrum of X-rays which have passed through a subject is a subject thickness in the irradiation direction. When the part to be radiographed is, for example, a hand, arm or leg, the thickness of the subject in the irradiation direction is substantially constant as long as a patient (subject) is not extremely fat or thin. Thus, identifying which part of a body the subject is can identify the subject thickness in the irradiation direction.

In view of this, the relationship between i) a subject thickness in the irradiation direction and/or which part of a body the subject is, and ii) the material and/or thickness of the member to create the change in energy spectrum of X-rays equivalent to the change in energy spectrum of X-rays created by the subject having such a thickness is obtained in advance.

Specifically, the change in energy spectrum of X-rays created by a subject having a given thickness in the irradiation direction is experimentally measured in advance, for example. The material and/or thickness of the member to create change in energy spectrum of X-rays equivalent to the change in energy spectrum of X-rays created by a subject with a certain thickness is specified while the material and/or thickness of a member is variously changed. This process is performed for each of different subject thicknesses in the irradiation direction to obtain the relationship in advance.

The relationship between a part of a body to be radiographed and the material and/or thickness of the member to create change in X-ray energy spectrum equivalent to the change in X-ray energy spectrum created by the part having a certain thickness may be obtained through computation using the widely-known physical property values of materials included in, for example, Rikagakujiten (Japanese dictionary on physics and chemistry, published by Iwanami), instead of the experimental creation of association through actual measurements.

Such a relationship may be obtained in advance by an announcement unit and stored therein. A radiation technologist etc. can input, to the announcement unit, the information on a subject thickness in the irradiation direction and/or the information on which part of a body the subject is. Alternatively, the announcement unit can obtain such information from a hospital information system (HIS) or a radiology information system (RIS). The announcement unit can then specify and announce the material and/or thickness of the member to be held in the background radiographing on the basis of the relationship.

The announcement unit may be provided in the radiographing apparatus 1, in which case the image processing apparatus 5 or the main body 18, for example, of the radiographing apparatus 1 (see FIG. 1) may be used as the announcement unit. Alternatively, announcement unit may be provided separately from the radiographing apparatus 1. The announcement unit gives information to radiation technologist etc. through an appropriate manner, such as display or voice.

Such a configuration allows the background radiographing to be performed while the member having the material and/or thickness announced by the announcement unit is held on the subject table 13 before or after the subject radiographing. The background radiographing creates change in energy spectrum of X-rays equivalent to the change in energy spectrum of X-rays created by the subject to produce the BG signal I_{BG} (x,y). The above-described calculation processing can be performed using the image signal I_{S} (x,y) obtained through the subject radiographing and the BG signal I_{BG} (x,y). This can generate an absorption image I_{AB} and small-angle scattering image I_{V} from which image disturbance has been surely removed.

The above-described configuration requires only one background radiographing for each subject radiographing, reducing power consumption and preventing the life of the X-ray source 11 from shortening.

In Example 2, the relationship between i) a subject thickness in the irradiation direction and/or which part of a body the subject is, and ii) the material and/or thickness of the member to create change in energy spectrum of X-rays equivalent to the change in energy spectrum of X-rays created by the subject is obtained in advance. Accurate measurement of a subject thickness, however, might be difficult in some cases.

The information on a subject thickness is reflected in the DC component I₀ of an image signal of moire fringes obtained through subject radiographing. The relationship between a radiographing condition, such as an mAs value (i.e., the product of tube current (mA) and time (sec)); the DC component I₀ of the image signal of moire fringes generated through radiographing of apart under the radiographing condition (e.g., mAs value); and the material and/or thickness of the member to create change in energy spectrum of X-rays equivalent to the change in energy spectrum of X-rays created by the subject may be obtained in advance. The material and/or thickness of a member may be obtained based on an mAs value (i.e., radiographing condition) at the time of radiographing of the subject, which part of a body the subject is, the DC component I₀ of generated moire fringes, and the relationship obtained in advance.

### Example 3

The method of Example 2 requires at least one background radiographing for each subject radiographing. As a practical matter, however, a radiation technologist etc. would not wish to perform the background radiographing for each subject radiographing.

In view of this, the image processing apparatus 5 may contain a plurality of BG signals I_{BG} (x,y) obtained in advance through multiple-time background radiographing performed with members made of different materials and/or having different thicknesses, and may select an appropriate BG signal. I_{BG} (x,y) depending on the situation. Such a configuration eliminates the need for performing background radiographing for each subject radiographing. Specific examples to achieve this are given below.

### Example 3-1

When performing multiple-time background radiographing with members having different materials and/or thicknesses as described above, the energy spectrum of X-rays which have passed through each member is also measured, and the BG signal I_{BG} (x,y) for each member is associated with a spectrum in advance.

The image processing apparatus 5 calculates the image signal I_{S} (x,y) of a moire image Mo obtained through subject radiographing, and estimates the energy spectrum of X-rays which have passed through the subject on the basis of the calculated image signal I_{S} (x,y). The image processing apparatus 5 then selects the spectrum equivalent to the estimated spectrum among the spectrums associated with the BG signals I_{BG} (x,y), and selects the BG signal I_{BG} (x,y) associated with the specified spectrum.

The image processing apparatus 5 then performs the calculation processing using the selected BG signal I_{BG} (x,y) and the image signal I_{S} (x,y) of the subject to generate an absorption image I_{AB} and small-angle scattering image I_{V} of the subject.

With such a configuration, the image processing apparatus 5 can surely and automatically select, among different BG signals I_{BG} (x,y) obtained in advance, the BG signal I_{BG} (x,y) obtained with the member having the material and/or thickness to create change in energy spectrum of X-rays equivalent to the change in energy spectrum of X-rays created by the subject; and can perform the calculation processing using the selected BG signal I_{BG} (x,y) and the image signal I_{S} (x,y) of the subject. This can generate an absorption image I_{AB} and small-angle scattering image I_{V} from which image disturbance has been surely removed.

If the spectrum equivalent to the energy spectrum of X-rays which have passed through the subject estimated on the basis of the image signal I_{S} (x,y) of the subject is not present in the energy spectrums of X-rays associated with the BG signals I_{BG} (x,y) obtained in advance, two spectrums which are closest to the estimated spectrum are extracted, and linear interpolation is performed for each pixel with the two BG signals I_{BG} (x,y) associated with the two spectrums, for example, to obtain the BG signal I_{BG} (x,y).

A plurality of BG signals I_{BG} (x,y) may be obtained through multiple-time background radiographing using members having different materials and/or thicknesses before actual radiographing by the radiographing apparatus 1 on the same day as the actual radiographing. Alternatively, the BG signals I_{BG} (x,y) may be obtained regularly (e.g., every few days or few months), or may be obtained at the time of calibration of the radiographing apparatus 1. The same applies to Examples 3-2 and 3-3 set forth below.

### Example 3-2

The image processing apparatus 5 may include in advance the relationship between i) a subject thickness in the irradiation direction and/or which part of a body the subject is, and ii) the material and/or thickness of the member to create change in energy spectrum of X-rays equivalent to the change in energy spectrum of X-rays created by the subject having such a thickness, as shown in Example 2.

In this case, the image processing apparatus 5 includes in advance the relationship between i) a subject thickness in the irradiation direction and/or which part of a body the subject is, and ii) the material/thickness of a member. The image processing apparatus 5 also includes a plurality of BG signals I_{BG} (x,y) obtained through the multiple-time background radiographing performed with the members having different materials and/or thicknesses.

When a radiation technologist etc. inputs the information on a subject thickness in the irradiation direction and/or the information on which part of a body the subject is or when the image processing apparatus 5 obtains such information from the HIS, RIS or the like, the image processing apparatus 5 specifies the material and/or thickness of the member to be held in the background radiographing on the basis of the relationship.

The image processing apparatus 5 determines, among the plurality of BG signals I_{BG} (x,y) obtained in advance, the BG signal I_{BG} (x,y) obtained through the background radiographing performed with the member having the specified material and/or thickness. The image processing apparatus 5 then performs the calculation processing using the selected BG signal I_{BG} (x,y) and the image signal I_{S} (x,y) of the subject to generate an absorption image I_{AB} and small-angle scattering image I_{V} of the subject.

With such a configuration, the image processing apparatus 5 can surely and automatically select, among different BG signals I_{BG} (x,y) obtained in advance, the BG signal I_{BG} (x,y) suitable for the obtained information on a subject thickness in the irradiation direction and/or on which part of a body the subject is; and can perform the calculation processing using the selected BG signal I_{BG} (x,y) and the image signal I_{S} (x,y) of the subject. This can generate an absorption image I_{AB} and small-angle scattering image I_{V} from which image disturbance has been surely removed.

### Example 3-31

In the above Examples 3-1 and 3-2, a plurality of BG signals I_{BG} (x,y) obtained through multiple-time background radiographing performed with the members having different materials and/or thicknesses are obtained in advance, and an absorption image I_{AB} and small-angle scattering image I_{V} are generated with the use of the BG signals I_{BG} (x,y).

With such a configuration, however, the positions of the first grating 14 and the second grating 15 may sometimes slightly change between the time of the background radiographing performed in advance and the time of the actual subject radiographing in some cases. Specifically, the change of the grating positions includes, for example, the change in the relative angle θ between the directions of the gratings 14 and 15, resulting in the change in periods of moire fringes in a moire image Mo and BG moire image Mb (i.e., the period of moire fringes of the moire image Mo represented by black and white in FIG. 3, for example).

Specifically, there may be change in period of moire fringes, in some cases, between a BG moire image Mb produced at the time of background radiographing performed in advance and a moire image Mo produced at the time of subject radiographing. The generated absorption image I_{AB} and small-angle scattering image I_{V} thus may be subject to the influence due to the difference in period of moire fringes. A differential phase image is reconstructed using the same BG image as that for an absorption image and a small-angle scattering image, and the expression (8) describes the case in which the relative angle θ formed by the directions of the first and second gratings 14 and 15 is the same between the subject radiographing and the BG radiographing. If the relative angle θ is different between the subject radiographing and the BG radiographing, an artifact may appear in the plane.

In view of this, image correction can be performed on the BG signal I_{BG} (x,y) selected by the image processing apparatus 5 in Examples 3-1 and 3-2, and the BG signal I_{BG} (x,y) after the image correction and the image signal I_{S} (x,y) of the subject can be used to generate an absorption image I_{AB} and small-angle scattering image I_{V} of the subject.

As described above, the present invention obtains the BG signal I_{BG} (x,y) through the background radiographing performed with a member held on the subject table 13, instead of performing the conventional background radiographing in which noting is held on the subject table 13. This applies to Example 3-3.

In Example 3-3, background radiographing is additionally performed with nothing held on the subject table 13 similarly to the conventional manner (i.e., with no subject and no member held on the subject table 13). In Example 3-3, the signal obtained through such background radiographing is additionally used as a reference signal.

Specifically, multiple-time background radiographing is first performed in advance with members made of different materials and/or having different thicknesses to produce a plurality of BG signals I_{BG} (x,y). At the same time as the acquisition of the BG signals I_{BG} (x,y), background radiographing is performed with nothing held on the subject table 13 to produce a signal. The signals obtained through the former multiple-time background radiographing are hereinafter referred to as BG_{S} signals I_{BGS} (x,y), and the signal obtained through the latter background radiographing is referred to as a BG_{N} signal I_{BGN} (x,y) to be distinguished from the above-described BG signals I_{BG} (x,y). The BG_{N} signal I_{BGN} (x,y) may be obtained every time the background radiographing is performed while the material and/or thickness of the member is changed. Alternatively, only one BG_{N} signal I_{BGN} (x,y) may be obtained for a series of the multiple-time background radiographing.

The image processing apparatus 5 stores in a storage unit the BG_{S} signals I_{BGS} (x,y) for the members made of different materials and/or having different thicknesses obtained through the multiple-time background radiographing and the BG_{N} signal I_{BGN} (x,y) such that the BG_{S} signals I_{BGS} (x,y) and the BG_{N} signal I_{BGN} (x,y) obtained at the same time are associated with each other.

As described above, the BG_{S} signals I_{BGS} (x,y) and the BG_{N} signal I_{BGN} (x,y) are obtained at the same time, and the same moire fringes appear at the same pixel position (x,y) between the BG_{S} signals I_{BGS} (x,y) and the BG_{N} signal I_{BGN} (x,y). Unlike the above-described Examples 3-1 and 3-2, the BG_{S} signals I_{BGS} (x,y) and the BG_{N} signal I_{BGN} (x,y) are stored in the storage unit in advance in this example.

When performing subject radiographing, background radiographing is also performed in the same manner as the above with nothing held on the subject table 13 (i.e., with no subject and no member held on the subject table 13) before or after the radiographing apparatus 1 radiographs the subject, to obtain the BG_{N} signal I_{BGN} (x,y).

The BG_{N} signal I_{BGN} (x,y) obtained in the subject radiographing is referred to as the BG_{N} signal I_{BGN} (x,y)_{NEW}, which means a BG_{N} signal I_{BGN} (x,y) obtained in the current radiographing. In this case, the BG_{N} signal I_{BGN} (x,y)_{NEW} includes a component of the moire fringes having a period determined depending on the relative angle θ between the directions of the first and second gratings 14 and 15 at the timing of the current radiographing.

The image processing apparatus 5 then selects one of the plurality of BG_{S} signals I_{BGS} (x,y) obtained in advance using the method described in Example 3-1 or 3-2. Specifically, the image processing apparatus 5 selects the BG_{S} signal I_{BGS} (x,y) obtained through the background radiographing performed with the member made of a specific material and/or having a specific thickness.

As described above, the selected BG_{S} signal I_{BGS} (x,y) includes a component of the moire fringes having a period determined depending on the relative angle θ between the directions of the first and second gratings 14 and 15 at the timing of the background radiographing for obtaining the BG_{S} signal I_{BGS} (x,y). As described above, there is a possibility that this period of the moire fringes differs from the period of the moire fringes included in the currently obtained BG_{N} signal I_{BGN} (x,y).

As shown in the expression (6), the image processing apparatus 5 performs the calculation processing in which the selected BG_{S} signal I_{BGS} (x,y), the BG_{N} signal I_{BGN} (x,y) associated with the selected BG_{S} signal I_{BGS} (x,y) (i.e., the BG_{N} signal I_{BGN} (x,y) obtained at the same time as the acquisition of the BG signal I_{BGS} (x,y)), and the BG_{N} signal I_{BGN} (x,y)_{NEW} currently obtained are each approximated by the sum of the DC component I₀ and the first-order amplitude component I₁ of the moire fringes. The component derived from the selected BG_{S} signal I_{BGS} (x,y) is divided by the component derived from the BG_{N} signal I_{BGN} (x,y) associated with the selected BG_{S} signal I_{BGS} (x,y), and the result is multiplied by the component derived from the currently obtained BG_{N} signal I_{BGN} (x,y)_{NEW}. Thus, the absorption signal I₀ (E_{BG0},x,y) and the small-angle scattering signal I₁ (E_{BG1},x,y)/I₀ (E_{BG0},x, y) of the BG signal corresponding to the spectrum change created due to the grating positions and the subject at the timing of the subject radiographing is obtained with the expressions (11) and (12), respectively.

I₀(E_{BG0},x,y)=I₀(E_{BGN}_{_{—}}_{NEW0},x,y)×(I₀(E_{BGS0},x,y)/I₀(E_{BGN0},x,y) (11)

I₁(E_{BG1},x,y)/I₀(E_{BG0},x,y)=(E_{BGN}_{_{—}}_{NEW1},x,y)/I₀(E_{BGN}_{_{—}}_{NEW0},x,y))×((I₁(E_{BGS1},x,y)/I₀(E_{BGS0},x,y))/(I₁(E_{BGN1}x,y)/I_{O}(E_{BGN0},x,y))) (12)

Then, the calculation processing is performed using the results obtained through the above calculation and the image signal I_{S} (x,y) obtained through the current subject radiographing to generate an absorption image I_{AB} and small-angle scattering image I_{V} of the subject, similarly to the above-described manner.

Thus, even when the moire fringes of the BG moire image Mb produced at the timing of the multiple-time background radiographing performed in advance are different in period from the moire fringes of the moire image Mo produced at the timing of the subject radiographing, the generated absorption image I_{AB} and small-angle scattering image I_{V} are surely free from the influence of such difference in moire fringe period.

Instead of performing the calculations by the above expressions (11) and (12) at the timing of the subject radiographing as described above, the terms derived from I_{BGS} (x,y) and I_{BGN} (x,y) in the expressions (11) and (12) may be calculated in advance at the timing of acquisition of BG_{S} signal I_{BGS} (x,y) and BG_{N} signal I_{BGN} (x,y), and the results may be stored in the storage unit in the form of corrected data r1 (x,y) and r2 (x,y) obtained by the expressions (13) and (14) shown below, for example.

r1(x,y)=I₀(E_{BGS0},x,y)/I₀(E_{BGN0},x,y) (13)

r2(x,y)=(I₁(E_{BGS1},x,y)/I₀(E_{BGS0},x,y))/(I₁(E_{BGN1}x,y)I₀(E_{BGN0}x,y)) (14)

The image processing apparatus 5 calculates the component derived from a BG signal corresponding to the spectrum change created due to the grating positions and the subject at the timing of the subject radiographing according to the expression (15), similar to the expression (13), in the case of an absorption signal.

I₀(E_{BG},x,y)=r1(x,y)×I₀(E_{BGN}_{_{—}}_{NEW0},x,y) (15)

The same applies to the terms of a small-angle scattering signal.

If the spectrum equivalent to the energy spectrum of X-rays which have passed through the subject estimated on the basis of the image signal I_{S} (x,y) of the subject is not present in the energy spectrums of X-rays associated with the various pieces of corrected data r1 obtained in advance, two spectrums which are closest to the estimated spectrum are extracted, and linear interpolation is performed for each pixel with the two pieces of corrected data r1 (x,y) associated with the two spectrums, for example, to obtain the corrected data r1 (x,y).

Alternatively, the relationships of correction values of various pieces of corrected data r1 provided in advance may be obtained to create a table, or the relationships and the relevant subject thicknesses may be made into a function. This enables calculation of the corrected data r1 corresponding to the spectrum of X-rays which have passed through a subject based on the corrected data r1 of the reference X-rays spectrum and the table or function.

In Examples 3-1, 3-2, and 3-3, an appropriate BG signal I_{BG} (x,y) is selected according to the image signal I_{S} (x,y) for each pixel, or corrected data is created using the selected BG signal I_{BG} (x,y) for correction. Alternatively, the BG image Mb suitable for the X-rays spectrum of the area of interest of the subject image Mo may be selected, or corrected data may be created using the selected BG image Mb for correction.

### Advantageous Effects

As described above, the medical imaging system according to this embodiment performs the background radiographing to obtain a BG signal I_{BG} (x,y) with the member having the material and/or thickness to create change in energy spectrum of X-rays equivalent to the change in energy spectrum of X-rays created by the subject, instead of conventional background radiographing in which nothing is held on the subject table 13. The image processing apparatus 5 performs background correction using the BG signal I_{BG} (x,y) obtained in this way and the image signal I_{S} (x,y) obtained thorough radiographing of the subject to generate an absorption image I_{AB} and small-angle scattering image I_{V} of the subject.

With the conventional system, the energy spectrum of X-rays which have passed through a subject is different from the energy spectrum of X-rays which have not passed through the subject (see FIG. 5), leading to difference in amount of X-rays passing through the first grating 14. This makes the degree of image disturbance included in the image signal I_{S} (x,y) different from that in the BG signal I_{BG} (x,y). The components of image disturbances, therefore, cannot offset with each other when the divisions shown in the expressions of (7) and (10) are performed for background correction. As a result, an image disturbance remains in an absorption image I_{AB} and small-angle scattering image I_{V} as shown in FIGS. 4A and 4B.

By contrast, the medical imaging system according to this embodiment performs background radiographing with the member having the material and/or thickness to create change in energy spectrum of X-rays equivalent to the change in energy spectrum of X-rays created by the subject to obtain the BG signal I_{BG} (x,y).

Thus, the energy spectrum of X-rays which have passed through the subject is equivalent to the energy spectrum of X-rays which have passed through the member (see FIG. 6), leading to the same or substantially the same amount of X-rays passing through the first grating 14. This makes the degree of image disturbance included in the image signal I_{S} (x,y) substantially the same as that in the BG signal I_{BG} (x,y). The components of image disturbances, therefore, surely offset with each other when the divisions shown in the expressions of (7) and (10) are performed for background correction.

As a result, an image disturbance is surely removed from an absorption image I_{AB} and small-angle scattering image I_{V} as shown in FIGS. 7A and 7B. In this way, the medical imaging system according to this embodiment can surely prevent an image disturbance, such as grating fringes and an artifact, from appearing in an absorption image I_{AB} and small-angle scattering image I_{V} reconstructed from a moire image Mo produced by the radiographing apparatus 1 provided with a Talbot interferometer or Talbot-Lau interferometer.

This surely prevents an absorption image I_{AB} and small-angle scattering image I_{V} from being fuzzy due to image disturbances remaining therein, and surely prevents inconvenience such as oversight of a lesion part of a patient which faintly appears in an image but mixed among the image disturbance.

[Processing to be Performed when Body Moves in Fringe Scanning]

As described above, examples of the methods of reconstructing an X-ray absorption image I_{AB}, differential phase image I_{DP}, and small-angle scattering image I_{V} on the basis of a moire image Mo produced with the radiographing apparatus 1 and a BG moire image Mb produced through the background radiographing include a method based on the principle of fringe scanning.

In the fringe scanning, when the second grating 15 (see FIG. 3) is scanned M times in the x direction, a subject is irradiated every time the second grating 15 is shifted by 1/M of its pitch d2, and M-time subject radiographing is performed. After that (or before that), background radiographing with no subject is performed M times while shifting the second grating 15 in the same manner. When the above-described embodiments are applied, the background radiographing is performed M times with a member made of a predetermined material and/or having a predetermined thickness held on the subject table 13.

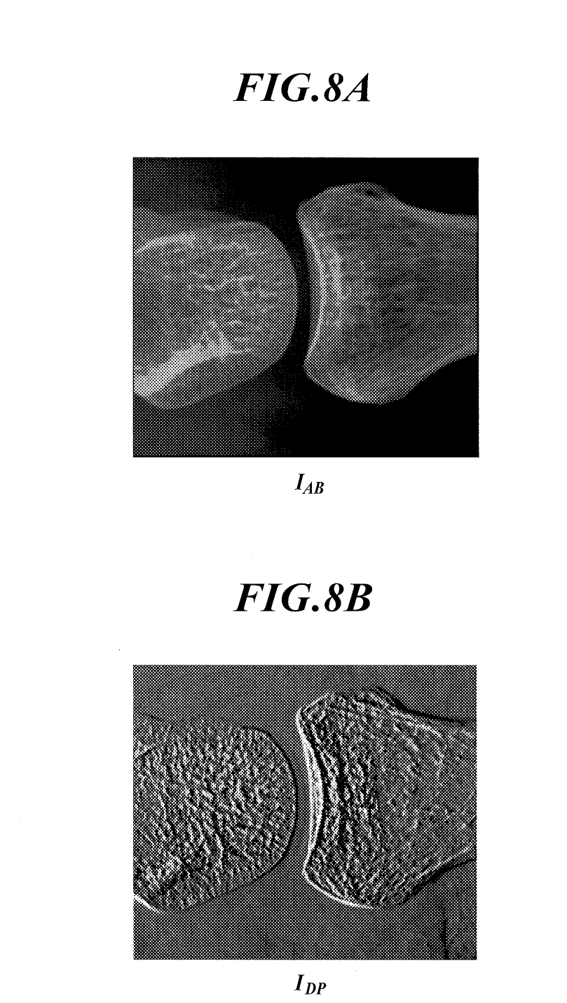

There may be a case in which a subject moves (i.e., a body movement occurs) during the M-time subject radiographing. With a small body movement of the subject, the outline etc. of the subject appears relatively sharply in an absorption image I_{AB} and differential phase image I_{DP} as shown in, for example, FIGS. 8A and 8B.

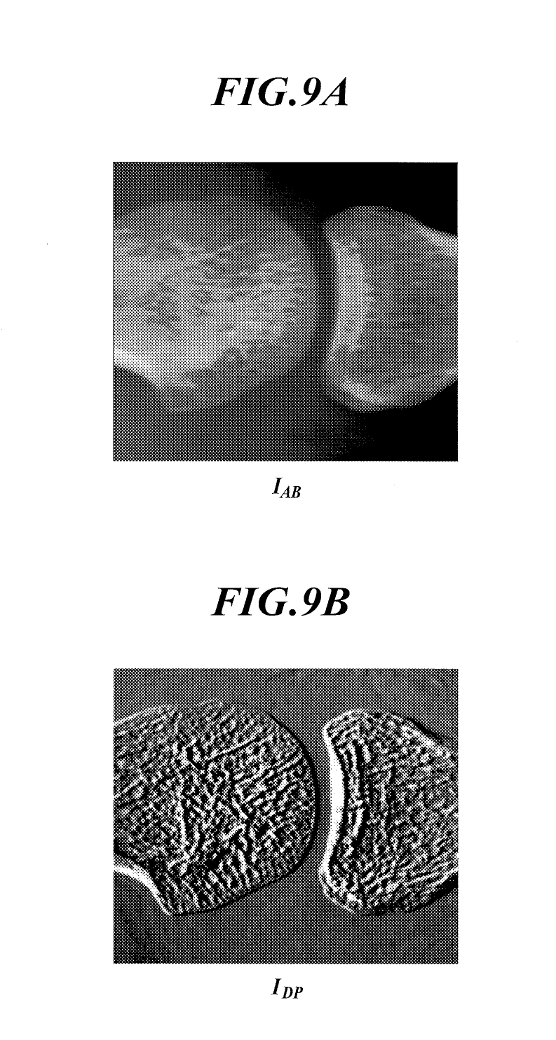

With a large body movement of the subject, by contrast, an absorption image I_{AB} and differential phase image I_{DP} are blurred as shown in, for example, FIGS. 9A and 9B. The same applies to a small-angle scattering image I_{V} although not shown in FIGS. 8A, 8B, 9A and 9B.

The following is the description of the processing for body movement correction in which presence or absence of a body movement and the direction of a body movement is determined using M image signals I_{S} (x,y,k) and M BG signals I_{BG}(x,y,k) obtained through a series of subject radiographing and background radiographing using the fringe scanning, where k is 0 to M−1 (see the expressions (5) and (6)). This processing is performed in the image processing apparatus 5 (see FIG. 1).

The basic concept of the processing set forth below is that a body movement made during M-time subject radiographing can be canceled or reduced by returning the image signals obtained through the subject radiographing after the body movement to the original positions by the amount of the body movement. Correction of the image signals etc. through such processing can change blurred images as shown in FIGS. 9A and 9B into images with sharp outlines etc. as shown in FIGS. 8A and 8B.

The case of M=2 is taken as an example here. Specifically, in this case, the 1^{st} subject radiographing is performed with the second grating 15 at the initial position, and then the second grating 15 etc. is moved (scanned) to perform the 2^{nd} subject radiographing. In this case, a body movement occurs between the 1^{st} and 2^{nd} subject radiographing.

A raw image signal I_{S}_{_{—}}_{RAW} (x,y,k) is obtained through each subject radiographing. Through the background radiographing performed for each subject radiographing, a row BG signal I_{BG}_{_{—}}_{RAW}(x,y,k) is obtained. The following processing is performed on the image signals I_{S}_{_{—}}_{RAW}(x,y,0) and I_{S}_{_{—}}_{RAW}(x,y,1); and on the BG signals I_{BG}_{_{—}}_{RAW}(x,y,0) and I_{BG}_{_{—}}_{RAW}(x,y,1).

With the four images, the calculation processing is normally performed according to the expressions (5) to (10) to generate an absorption image I_{AB} differential phase image I_{DP}, and small-angle scattering image I_{V}. The generated absorption image I_{AB} etc. is represented as an absorption image I_{AB}(0) etc.

Next, the image signal I_{S}_{_{—}}_{RAW}(x,y,1) and the BG signal I_{BG}_{_{—}}_{RAW}(x,y,1) are translated in a predetermined direction relative to the image signal I_{S}_{_{—}}_{RAW}(x,y,0) and the BG signal I_{BG}_{_{—}}_{RAW}(x,y,0), respectively. In the following description, the predetermined direction is the x direction. The following description, however, applies to the case in which the predetermined direction is the y direction.

In the following description, the processing is performed on the row image signal I_{S}_{_{—}}_{RAW}(x,y,k) and row BG signal I_{BG}_{_{—}}_{RAW} (x,y,k). Alternatively, the processing may be performed on the image signal I_{S}(x,y,k) and BG signal I_{BG}(x,y,k) each approximated by the sum of the DC component I₀ and the first-order amplitude component I₁ of moire fringes (see the expressions (5) and (6)).

In the following description, although the processing on the BG signal I_{BG}_{_{—}}_{RAW}(x,y,k) is sometimes omitted, it is to be understood that, when the processing is performed on the image signal I_{S}_{_{—}}_{RAW}(x,y,k), the processing on the corresponding BG signal I_{BG}_{_{—}}_{RAW}(x,y,k) is similarly performed.

In this processing, the image signal I_{S}_{_{—}}_{RAW}(x,y,1) obtained through the 2^{nd} subject radiographing is translated by one pixel in the x direction (i.e., the predetermined direction) relative to the image signal I_{S}_{_{—}}_{RAW}(x,y,0) obtained through the 1^{st} subject radiographing, so as to create the image signal I_{S}_{_{—}}_{RAW}(x,y,1)(x:+1). The BG signal I_{BG}_{_{—}}_{RAW}(x,y,1) is also translated by one pixel in the x direction relative to the BG signal I_{BG}_{_{—}}_{RAW}(x,y,0), so as to create the BG signal I_{BG}_{_{—}}_{RAW}(x,y,1)(x:+1).

The calculation processing is performed according to the expressions (5) to (10) on the image signal I_{S}_{_{—}}_{RAW}(x,y,0) and the created image signal I_{S}_{_{—}}_{RAW}(x,y,1)(x:+1) to generate an absorption image I_{AB}, differential phase image I_{DP} and small-angle scattering image I_{V}. The generated absorption image I_{AB} etc. is represented as an absorption image I_{AB}(x:+1) which means an image obtained on the basis of the image signal etc. translated by one pixel in the x direction for the position correction.

In the same manner, the image signal I_{S}_{_{—}}_{RAW} (x,y,1) is translated by two pixels in the x direction relative to the image signal I_{S}_{_{—}}_{RAW}(x,y,0) to create the image signal I_{S}_{_{—}}_{RAW} (x,y,1)(x:+2). The calculation processing is then performed on the image signal I_{S}_{_{—}}_{RAW}(x,y,0) and the created image signal I_{S}_{_{—}}_{RAW} (x,y,1)(x:+2) according to the expressions (5) to (10) to generate an absorption image I_{AB}(x:+2), differential phase image I_{DP}(x:+2), and small-angle scattering image I_{V}(x:+2).

The same processing is repeated subsequently. That is, the image signals I_{S}_{_{—}}_{RAW}(x,y,1) are sequentially translated by n pixels in the x direction relative to the image signal I_{S}_{_{—}}_{RAW}(x,y,0) to create image signals I_{S}_{_{—}}_{RAW}(x,y,1)(x:+n). Each time an image signal I_{S}_{_{—}}_{RAW} (x,y,1)(x:+n) is created, the calculation processing is performed on the image signal I_{S}_{_{—}}_{RAW}(x,y,0) and the created image signal I_{S}_{_{—}}_{RAW} (x,y,1)(x:+n) according to the expressions (5) to (10). Absorption images I_{AB}(x:+n), differential phase images I_{DP}(x:+n), and small-angle scattering images I_{V}(x:+n) are thus sequentially generated.

The same processing is performed for the opposite direction, i.e., the minus direction with respect to the x direction (i.e., the predetermined direction).

Specifically, the image signals I_{S}_{_{—}}_{RAW} (x,y,1) are sequentially translated in the x direction by (−n) pixels relative to the image signal I_{S}_{_{—}}_{RAW}(x,y,0) to create the image signals I_{S}_{_{—}}_{RAW}(x,y,1)(x:−n). Each time an image signal I_{S}_{_{—}}_{RAW}(x,y,1)(x:−n) is created, the calculation processing is performed on the image signal I_{S}_{_{—}}_{RAW}(x,y,0) and the created image signal I_{S}_{_{—}}_{RAW}(x,y,1)(x:−n) according to the expressions (5) to (10). Absorption images I_{AB}(x:−n), differential phase images I_{DP}(x:−n), and small-angle scattering images I_{V}(x:−n) are thus sequentially generated.

During multiple-time continuous subject radiographing in fringe scanning, a subject patient is told by a radiation technologist etc. to keep his/her body still, and a large body movement will not occur if any. For this reason, several or ten-something pixels at most is enough as the translation distance of the image signal I_{S}_{_{—}}_{RAW}(x,y,1) in the predetermined direction relative to the image signal I_{S}_{_{—}}_{RAW}(x,y,0).

The absorption image I_{AB}(x:n*) etc. with the sharpest outline etc. is selected, as the image after the body movement correction processing, from the generated absorption images I_{AB} (x:±n) etc. The body movement correction processing in the fringe scanning is thus performed. A method for selecting a specific absorption image I_{AB}(x:n*) etc. from the multiple absorption images I_{AB}(x:±n) etc. is described later.

If the selected image after the body movement correction processing is an absorption image I_{AB}(0), differential phase image I_{DP}(0), or small-angle scattering image I_{V}(0), it is determined that a body movement of a subject has not occurred during the multiple-time subject radiographing in the fringe scanning. If the selected image after the body movement correction processing is an absorption image I_{AB}(x:n*) etc. (n*≠0), it is determined, from the plus or minus of n* and its absolute value, which of the plus and minus directions with respect to the x direction (i.e., the predetermined direction) and to what degree the subject has moved during the multiple-time subject radiographing in the fringe scanning.

[Method of Selecting Specific Image from Multiple Generated Images]

### [Selecting Method 1]

Examples of the methods of selecting a specific absorption image I_{AB}(x:n*) etc. from the multiple generated absorption images I_{AB}(x:±n) etc. in the body movement correction processing include selecting the absorption image I_{AB}(x:n*) etc. with a sharpest subject outline etc.

In this case, a bone edge can be specified in an image, such as an absorption image I_{AB} shown in FIGS. 8A and 9A and a differential phase image I_{DP} shown in FIGS. 8B and 9B (the same applies to a small-angle scattering image I_{V} not shown).

Specifically, in each pixel row (i.e., each pixel row with one-pixel width extending in the right-left direction or x direction of the image) of an absorption image I_{AB}(x:±n) as shown in FIGS. 8B and 9A, the differences between the signal values I_{AB} (x,y) of adjacent pixels are calculated (see the expression (7)).

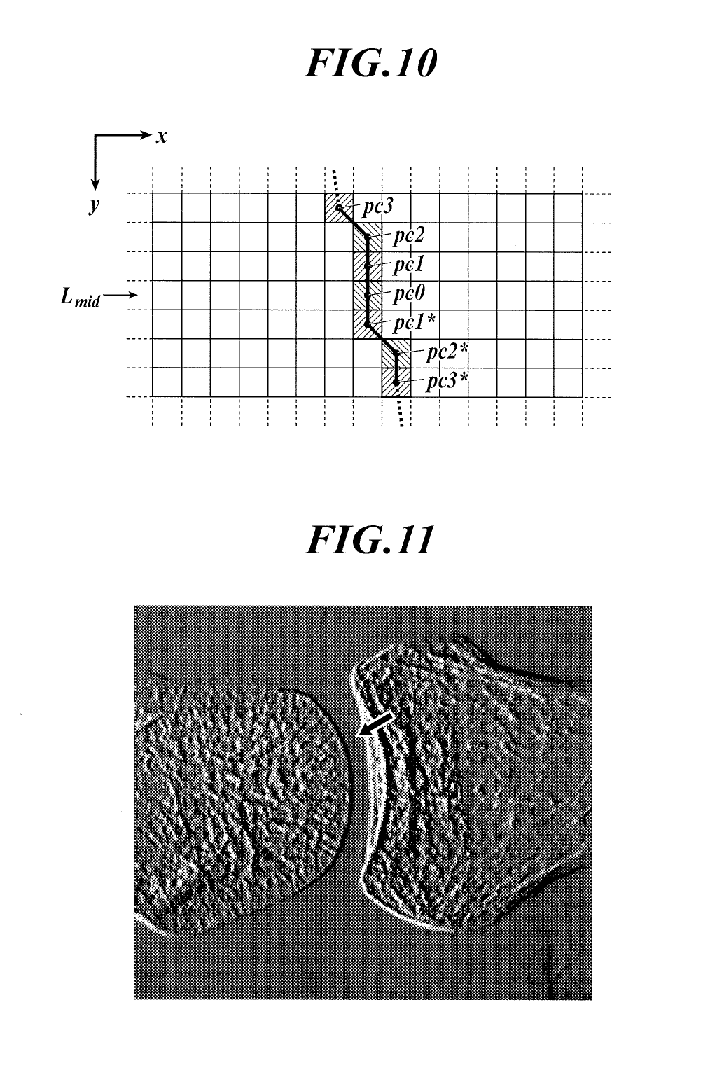

The pixels for which the absolute values of the calculated differences are equal to or more than a predetermined threshold are marked. As shown in FIG. 10, a continuously arranged marked pixels . . . , pc3, pc2, pc1, pc0, pc1*, pc2*, and pc3* . . . , appear. Such a part can be specified as the position of the bone edge in an absorption image I_{AB}(x:±n) etc.

Checking the degrees of decrease or increase in signal value I_{AB} (x,y) from each pixel pc0 etc. (corresponding to a specified bone edge) to the pixels to its right and left enables acquisition of the sharpness of the image. Specifically, as the increase or decrease in signal value I_{AB} (x,y) from each pixel pc0 etc. (corresponding to a bone edge) to the pixels to its right and left is steeper, the image is determined to be sharper.

### [Selecting Method 2]

Instead of checking the slopes (i.e., the degrees of decrease or increase) of the signal values I_{AB} (x,y) of the pixels at a bone edge part, the sharpness of the image may be determined depending on the difference between the maximum and minimum of the signal values I_{AB} (x,y) at the part. In this case, as the difference between the maximum and minimum of the signal values I_{AB} (x,y) is larger, the image is determined to be sharper.

### [Selecting Method 3]

The studies conducted by the inventors of the present invention have found that a cartilage edge present between the two bones constituting a joint appears in a sharp differential phase image I_{DP} created from a moire image Mo of the joint, as indicated by the arrow in FIG. 11.

How sharply the cartilage edge appears can be used as an index of sharpness of a differential phase image I_{DP}.

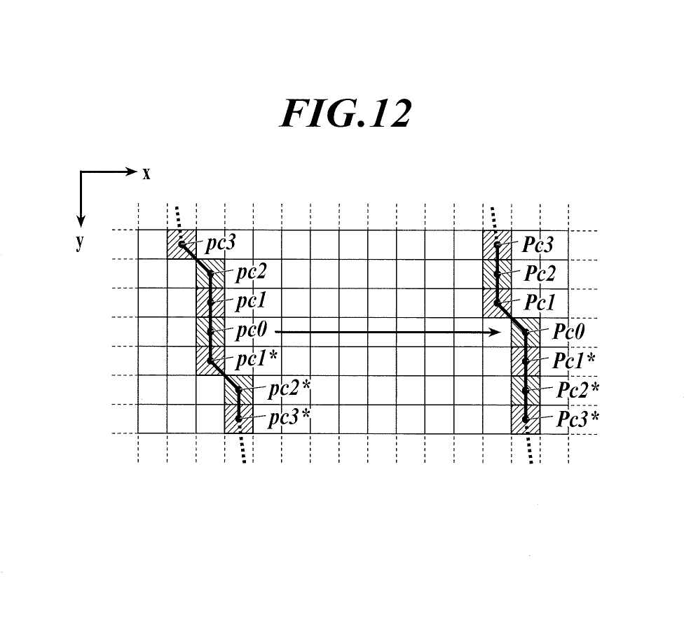

In this case, the position of cartilage edge can be specified on the basis of the bone edge specified as described above. Specifically, the differences in signal value I_{DP} (x,y) between each of the pixels . . . , pc3, pc2, pc1, pc0, pc1*, pc2*, and pc3*, . . . (corresponding to the specified bone edge) and the pixels to its right and left are calculated, and the pixels . . . , Pc3, Pc2, Pc1, Pc0, Pc1*, Pc2*, Pc3*, . . . for which the absolute values of the calculated differences are equal to or more than a predetermined threshold are detected as a cartilage edge, as shown in FIG. 12.

In this case, too, the sharpness of at least a differential phase image I_{DP} can be determined by checking the degrees of decrease or increase in signal value I_{DP} (x,y) from each pixel Pc0 etc. (corresponding to the specified cartilage edge) to the pixels near the pixel Pc0 etc., or by calculating the difference between the maximum and minimum of the signal values I_{DP} (x,y) at that part.

The description above focuses on sharpness of an image, and the sharpest absorption image I_{AB}(x:n*) etc. is selected as a specific absorption image I_{AB}(x:n) etc. from the generated multiple absorption images I_{AB}(x:±n) etc. in the body movement correction processing.

Instead of or in addition to that, a specific absorption image I_{AB}(x:n*) etc. may be selected from the generated multiple absorption images I_{AB}(x:±n) etc. in the following method, for example.

As described above, when a large body movement of a subject occurs in the multiple-time subject radiographing using the fringe scanning, an absorption image I_{AB} of the subject is blurred as shown in, for example, FIG. 9A. This means that the differences between signal values I_{AB} (x,y) in an absorption image I_{AB} shown in FIG. 9A are smaller than those in FIG. 8A, a sharper image, which is subject to a less body movement.

Specifically, with a small body movement as shown in FIG. 8A, the differences between white and black parts are clear in an absorption image I_{AB}; while with a large body movement as shown in FIG. 9A, both of white and black parts in an absorption image I_{AB} look gray (i.e., a color close to intermediate colors), making the whole image grayish.

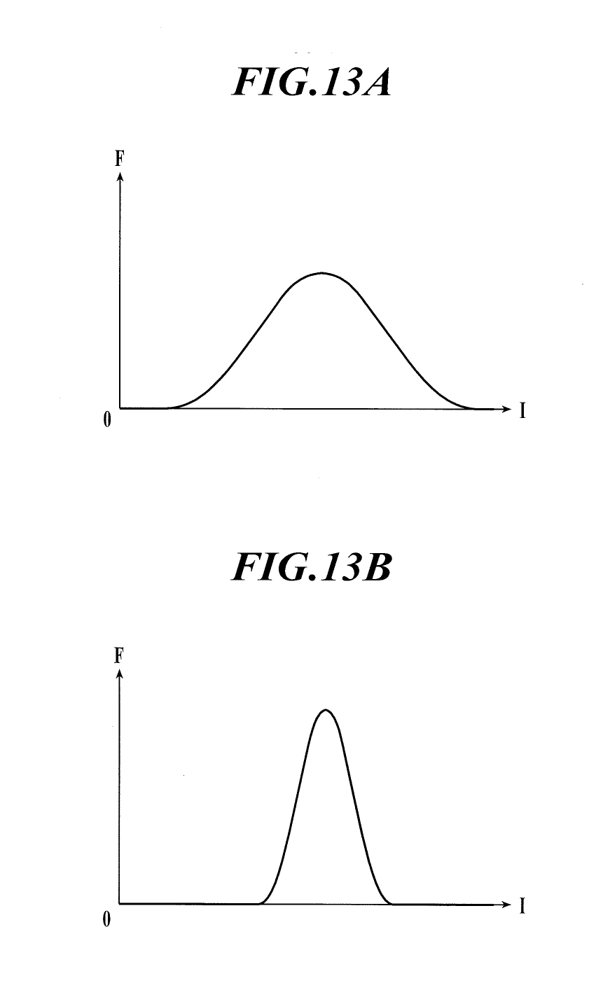

In view of this, each time an absorption image I_{AB}(x:±n) etc. is generated, the signal value I of the generated image is given to a histogram during the sequential image generation. When a body movement is small as shown in FIG. 8A, the differences between white and black parts are clear in the image, where pixels with high signal values I and low signal values I are present. This leads to wide distribution of the frequency F as shown in FIG. 13A.

By contrast, when a body movement is large as shown in FIG. 9A, both white and black parts in the image come close to intermediate colors, which makes the whole image grayish. Many of the signal values I thus are close to intermediate colors. This leads to narrow distribution of the frequency F as shown in FIG. 13B.

In view of the above, a histogram is created for each of the generated multiple images, and signal values I of the images are given to their respective histograms. The distribution widths of frequency F are compared with one another through the calculations of their standard deviations σ or variances σ². The image having the widest distribution of frequency F may be selected as a specific absorption image I_{AB}(x:n) etc. from the generated multiple absorption images I_{AB}(x:±n) etc. in the body movement correction processing. The image for which such histograms are created is not limited to an absorption image.

### [Modification of Body Movement Correction Processing]

In the above-described body movement correction processing, the image signal I_{S}_{_{—}}_{RAW} (x,y,1) obtained through the 2^{nd} subject radiographing is translated in the predetermined direction (for example, in the x direction) relative to the image signal I_{S}_{_{—}}_{RAW}(x,y,0) obtained through the 1^{st} subject radiographing. With the number of pixels by which the signal is translated variously changed, image signals I_{S}_{_{—}}_{RAW}(x,y,1)(x:±n) are created. The calculation processing is then preformed on the image signal I_{S}_{_{—}}_{RAW}(x,y,0) and the created image signals I_{S}_{_{—}}_{RAW}(x,y,1)(x:±n) according to the expressions (5) to (10) to sequentially generate absorption images I_{AB}(x:±n) etc. A specific one of the generated multiple absorption images I_{AB}(x:±n) etc. is then selected.

### [Modification 1]

The above-described method may be extended so that the image signal I_{S}_{_{—}}_{RAW}(x,y,1) obtained through the 2^{nd} subject radiographing is translated relative to the image signal I_{S}_{_{—}}_{RAW}(x,y,0) obtained through the 1^{st} subject radiographing two-dimensionally, instead of one-dimensionally (e.g., only x or y direction).

In this case, the image signal I_{S}_{_{—}}_{RAW} (x,y,1) obtained through the 2^{nd} subject radiographing is translated by i pixels in the x direction and by j pixels in the y direction relative to the image signal I_{S}_{_{—}}_{RAW} (x,y,0) obtained through the 1^{st} subject radiographing. The image signal after the translation is represented as I_{S}_{_{—}}_{RAW}(x,y,1)(x:i,y:j). The values i and j (including negative values) are determined within a predetermined range for two-dimensional translation, and thus image signals I_{S}_{_{—}}_{RAW} (x,y,1)(x:y:j) are sequentially created.

Each time an image signal I_{S}_{_{—}}_{RAW}(x,y,1)(x:i,y:j) is created, the calculation processing is performed on the image signal I_{S}_{_{—}}_{RAW} (x,y,0) and the created image signal I_{S}_{_{—}}_{RAW}(x,y,1)(x:i,y:j) according to the expressions (5) to (10) to sequentially generate absorption images I_{AB}(x:i,y:j) etc. A specific one of the generated multiple absorption images I_{AB}(x:i,y:j) etc. may be selected in the same manner as the above.

Such a configuration enables an accurate grasp of a two-dimensional body movement of a subject and enables appropriate body movement correction processing on absorption images I_{AB} etc.

### [Modification 2]

In the body movement correction processing described above, the number M of fringe scanning is 2 for ease of explanation. Specifically, the 1^{st} subject radiographing is performed with the second grating 15 at the initial position, and then the second grating 15 is moved (scanned) to perform the 2^{nd} subject radiographing. Actually, however, the number M of fringe scanning is set to a larger number so that the second grating 15 is moved (scanned) for subject radiographing more than twice in many cases.

In this case, the above-described one-dimensional or two-dimensional body movement correction processing may be performed in a round-robin manner, as it were.

An example of one-dimensional body movement correction processing is taken here. Pixel numbers are set by which the image signals I_{S}_{_{—}}_{RAW}(x,y,1), I_{S}_{_{—}}_{RAW}(x,y,2), . . . , and I_{S}_{_{—}}_{RAW}(x,y,M−1) obtained through the 2^{nd},3^{rd}, . . . , and M^{th} subject radiographing, respectively, are translated relative to the image signal I_{S}_{_{—}}_{RAW}(x,y,0) obtained through the 1^{st} subject radiographing. Absorption images I_{AB}(x:±n) etc. are then generated in the same manner as the above. A specific one of the generated multiple absorption images I_{AB} etc. may be selected.

Such body movement correction processing enables an accurate grasp of any body movement of a subject occurring in the multiple-time subject radiographing using the fringe scanning, enables appropriate body movement correction, and enables selection of a sharper absorption image I_{AB} etc.

### [Modification 3]

The studies conducted by the inventors of the present invention found that a body movement of a subject is not constantly occurring (i.e., the subject is not constantly moving) during the multiple-time subject radiographing using the fringe scanning, but that a slight body movement occurs momentarily and suddenly in most cases.

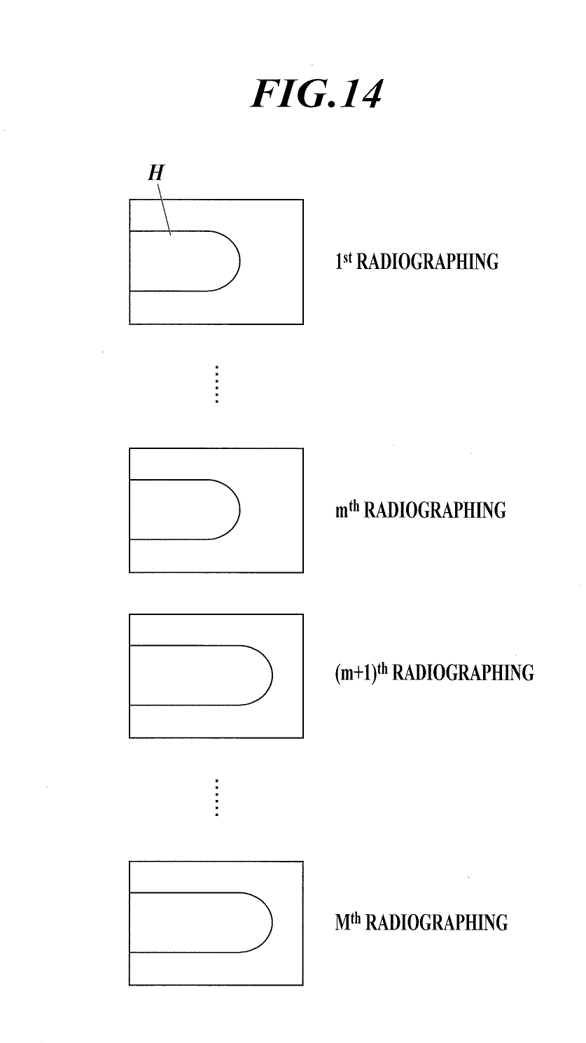

Specifically, it has been found that, as shown as typical diagrams in FIG. 14, a body movement of a subject H does not occur from the 1^{st} to m^{th} subject radiographing, occurs between the m^{th} and (m+1)^{th} subject radiographing, and does not occur from the (m+1)^{th} to M^{th} subject radiographing, for example.

With the use of this knowledge, more practical and easier body movement correction processing can be performed instead of performing an immense amount of calculation processing through the round-robin body movement correction processing.

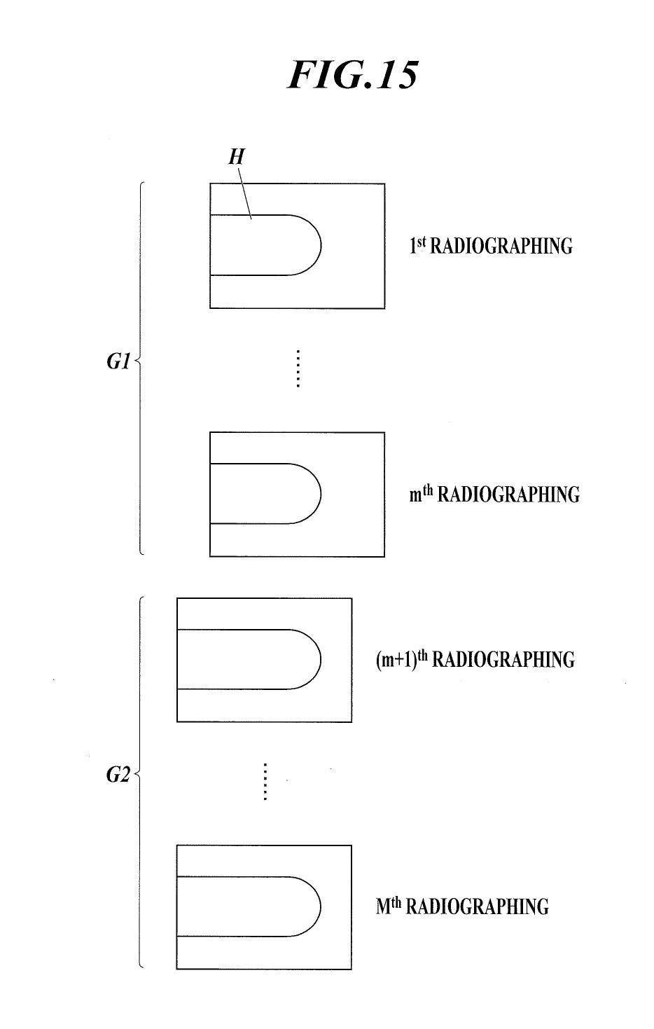

Specifically, M image signals I_{S}_{_{—}}_{RAW} (x,y,0) to I_{S}_{_{—}}_{RAW}(x,y,M−1) obtained through the 1^{st} to M^{th} subject radiographing are divided into groups G1 and G2. The group G1 includes the image signals obtained through the 1^{st} to m^{th} subject radiographing, and the group G2 includes the image signals obtained through the (m+1)^{th} to M^{th} subject radiographing, as shown in FIG. 15.

The image signals belonging to the group G1, i.e., the image signals I_{S}_{_{—}}_{RAW} (x,y,1) to I_{S}_{_{—}}_{RAW}(x,y,m−1) obtained through the 2^{nd} to m^{th} subject radiographing are not translated relative to the image signal I_{S}_{_{—}}_{RAW}(x,y,0) obtained through the 1^{st} subject radiographing. The image signals belonging to the group G2, i.e., the image signals I_{S}_{_{—}}_{RAW}(x,y,m) to I_{S}_{_{—}}_{RAW}(x,y,M−1) obtained through the (m+1)^{th} to M^{th} subject radiographing are translated simultaneously by the same number of pixels relative to the image signal I_{S}_{_{—}}_{RAW} (x,y,0) obtained through the 1^{st} subject radiographing. At this time, the (m+1)^{th} to M^{th} image signals I_{S}_{_{—}}_{RAW}(x,y,m) to I_{S}_{_{—}}_{RAW}(x,y,M−1) are not translated relative to each other.

The calculation processing is then performed on the 1^{st} to M^{th} image signals I_{S}_{_{—}}_{RAW}(x,y,0) to I_{S}_{_{—}}_{RAW}(x,y,M−1) according to the expressions (5) to (10) to generate an absorption image I_{AB}, differential phase image I_{DP} and small-angle scattering image I_{V}.

The number “m”, which is a parameter for dividing M image signals into two groups G1 and G2, is varied within the range from 1 to M−1, and the number of pixels by which the image signals I_{S}_{_{—}}_{RAW}(x,y,m) to I_{S}_{_{—}}_{RAW}(x,y,M−1) belonging to the group G2 (i.e., the (m+1)^{th} to M^{th} image signals) are simultaneously translated relative to the 1^{St} image signal I_{S}_{_{—}}_{RAW}(x,y,0) is varied within a predetermined range. An absorption image I_{AB}, differential phase image I_{DP}, and small-angle scattering image I_{V} are generated for each case.

Selecting a sharper image using any of the selecting methods 1-3, for example, from the generated absorption images I_{AB} etc. enables the body movement correction processing on the absorption image I_{AB} etc., determination of presence or absence of a body movement of a subject, and determination of in which direction and to what degree a body movement of a subject has occurred.

In the body movement correction processing etc., the only element that moves in an absorption image I_{AB} etc. is a subject, and a background does not move at the time of a body movement of the subject. The target range of the image correction by the body movement correction processing may be limited to an area of interest including the subject area, and the body movement correction processing does not necessarily have to be performed on the background area.

It should be understood that the present invention is not limited to the embodiments but may be modified as appropriate without departing from the spirit of the present invention.

The entire disclosure of Japanese Patent Application No. 2013-005047 filed on Jan. 16, 2013 including description, claims, drawings, and abstract are incorporated herein by reference in its entirety.

Although various exemplary embodiments have been shown and described, the invention is not limited to the embodiments shown. Therefore, the scope of the invention is intended to be limited solely by the scope of the claims that follow.

What is claimed is:

1. A medical imaging system comprising:
a radiographing apparatus provided with a Talbot interferometer or a Talbot-Lau interferometer, the radiographing apparatus including:
an X-ray source which emits X-rays,
an X-ray detector including a conversion element to generate an electrical signal according to the emitted X-rays, and reading the electrical signal generated by the conversion element, as an image signal, and
a subject table to hold a subject; and
an image processing apparatus which generates at least one of an X-ray absorption image, a differential phase image, and a small-angle scattering image of the subject on the basis of the image signal obtained through subject radiographing in which the subject is radiographed by the radiographing apparatus, wherein
the image processing apparatus generates at least one of the X-ray absorption image, the differential phase image, and the small-angle scattering image of the subject using the image signal and a background signal obtained through the subject radiographing and background radiographing, respectively, the background radiographing being performed with a member held instead of the subject, the member having a material and/or thickness to create change in energy spectrum of X-rays equivalent to change in energy spectrum of X-rays created by the subject.

2. The medical imaging system according to claim 1, wherein
the background radiographing is performed multiple times with members having different materials and/or thicknesses to produce background signals, and the image processing apparatus obtains the background signals in advance; and
on the basis of the image signal obtained through the subject radiographing, the image processing apparatus selects, from the background signals, the background signal obtained with the member having the material and/or thickness to create the change in energy spectrum of X-rays equivalent to the change created by the subject, and uses the selected background signal.

3. The medical imaging system according to claim 2, wherein
the image processing apparatus obtains in advance a relationship between i) a subject thickness in an irradiation direction and/or which part of a body the subject is, and ii) the material and/or thickness of the member to create the change in energy spectrum of X-rays equivalent to the change created by the subject; and
when the image processing apparatus obtains information on the subject thickness in the irradiation direction and/or information on which part of the body the subject is, the image processing apparatus specifies the optimum material and/or thickness of the member on the basis of the relationship; selects the background signal obtained with the member having the specified material and/or thickness or having the material and/or thickness closest to the specified material and/or thickness; and uses the selected background signal.

4. The medical imaging system according to claim 2, wherein
the image processing apparatus obtains in advance a relationship between i) a radiographing condition for the subject radiographing, which part of a body the subject is, and the image signal for a specific part of an image of the subject, and ii) the material and/or thickness of the member to create the change in energy spectrum of X-rays equivalent to the change created by the subject; and
the image processing apparatus specifies the optimum material and/or thickness of the member on the basis of the radiographing condition for the subject radiographing, which part of the body the subject is, the image signal for the specific part of the image of the subject, and the relationship; selects the background signal obtained with the member having the specified material and/or thickness or having the material and/or thickness closest to the specified material and/or thickness; and uses the selected background signal.

5. The medical imaging system according to claim 2, wherein
before or after the radiographing apparatus performs the subject radiographing, the X-ray source emits X-rays with neither the subject nor the member held on the subject table under a same radiographing condition as a radiographing condition for the subject radiographing to produce a signal to be read by the X-ray detector; and
the image processing apparatus corrects the selected background signal using the produced signal and uses the corrected background signal.

6. The medical imaging system according to claim 1, further comprising an announcement unit which obtains in advance a relationship between i) a subject thickness in an irradiation direction and/or which part of a body the subject is, and ii) the material and/or thickness of the member to create the change in energy spectrum of X-rays equivalent to the change created by the subject, wherein
when the announcement unit obtains information on the subject thickness in the irradiation direction and/or information on which part of the body the subject is, required for specifying the material and/or thickness of the member on the basis of the relationship, the announcement unit specifies, on the basis of the relationship, the material and/or thickness of the member to be held in the background radiographing, and announces the specified material and/or thickness.
